(19) United States
(12) Patent Application Publication
(10) Pub. No.: US 2010/0126880 A1
(43) Pub. Date: May 27, 2010

(54) DNA COMPLEXING AGENTS

(76) Inventors: Hsiao-Hua Yu, Singapore (SG); Jackie Y. Ying, Singapore (SG); Natalia C. Tansil, Singapore (SG); Eric Assen B. Kantchev, Singapore (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/514,299

(22) PCT Filed: Nov. 9, 2007
(86) PCT No.: PCT/SG2007/000384

§ 371 Date: Feb. 8, 2010

Related U.S. Application Data
(60) Provisional application No. 60/865,228, filed on Nov. 10, 2006.

(51) Int. Cl. C07D 471/04; C07F 15/00; C08F 26/06; G01N 27/00; G01N 27/26

(52) U.S. Cl. 205/775; 546/2; 546/66; 526/259; 526/262; 324/71.1

(57) ABSTRACT

The invention provides a compound of structure (I): wherein X is S, O or NR^{N}, where R^{N} is H or alkyl; L is a linker group; Q is a group capable of binding with dsDNA; G and G′ are, independently, absent or have between 1 and 20 main chain atoms; FG is a functional moiety comprising at least one O or N atom or a transition metal complex; and R is selected from the group consisting of H, alkyl, alkoxy or OCR^{a}R^{b} coupled to an atom in L so as to form a six-membered ring. R^{a} and R^{b} are independently H or optionally substituted alkyl.

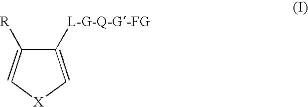

### TECHNICAL FIELD

The present invention relates to compounds and polymers thereof capable of complexing with double strand DNA and to the use thereof for detecting polynucleotides.

### BACKGROUND

DNA biosensors are one of the most promising tools for molecular diagnostics. The majority of protocols for sequence-specific DNA detection required prior labeling of the target DNA, but effective DNA labeling procedures are limited and require intensive washing protocols to prevent non-specific labeling. Intercalators are popular for nucleic acid detection due to their selective binding with double-stranded DNA (dsDNA) after hybridization of the label-free target DNA with the capture probe. These intercalators generally couple an intercalating unit with a small molecule or biomolecule that is capable of generating electrical or optical signals. Electrochemical intercalators are of particular interest because electrochemical detection is more cost-effective and capable of rapid, direct, and light-absorbing-tolerant detections. In addition, the detection devices are built with portable, robust, low-cost and easy-to-handle electrical components, so electrochemical detection is suitable for field tests and point-of-care use. Integrating electrochemical intercalators with electrocatalytic reactions has been shown to amplify the amperometric output and lower the DNA detection limit. Intercalating organic/inorganic compounds that bind selectively and reversibly to double-stranded DNA (dsDNA) have demonstrated applications as antitumor drugs, DNA probes and gene delivery vectors. Fluorescent and redox-active intercalators are employed as indicators for DNA hybridization to avoid labeling of target DNA. Complementary DNA targets are detected measuring optical/electrochemical outputs from these intercalators. However, most of these methods are limited by low signal intensity and poor signal/noise ratio.

Thus, there is an ongoing need in the art for improved signal output and reduced detection limit by using conducting polymers, such as those based on ethylenedioxythiophene (EDOT) monomers, coupled with intercalating units for DNA binding.

### SUMMARY OF THE INVENTION

In a first aspect of the invention there is provided a compound of structure (I)

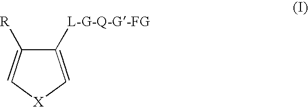

wherein:

X is S, O or NR^{N}, where R^{N} is H or alkyl;

L is a linker group;

Q is a group capable of binding with double-stranded DNA;

G and G′ are, independently, absent or have between 1 and 30 main chain atoms and, if present, are optionally substituted;

FG is a functional moiety comprising at least one O or N atom or a transition metal complex; and

R is selected from the group consisting of H, alkyl, alkoxy or OCR^{a}R^{b} coupled to an atom in L so as to form a six-membered ring, wherein R^{a} and R^{b} are independently H or optionally substituted alkyl.

The following options may be used in conjunction with the first aspect, either individually or in any appropriate combination.

The compound of structure (I) may be a symmetrical compound. It may be an asymmetric compound.

X may be S.

Q may be capable of selectively binding with the dsDNA. It may be capable of intercalating the dsDNA. It may be capable of binding the dsDNA by a threading intercalation mode. Q may be a naphthalene diimide group.

L may have structure OCH₂.

The compound of structure (I) may have structure (II).

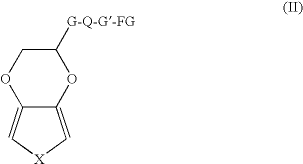

It may comprise a 3,4-ethylenedioxythiophene group coupled to a G-Q-G′-FG group, where G, G′, Q and FG are as defined earlier.

G and G′ may be independently selected from the group consisting of CH₂, (CH₂)₃, CH₂O(CH₂)₆, CH₂OCH₂(CH₂OCH₂)₂CH₂, CH₂OCH₂(CH₂OCH₂)₄CH₂ and CH₂OCH₂(CH₂OCH₂)₃CH₂.

FG may be OH, NH₂, imidazolyl, pyridinyl or a transition metal complex e.g. an osmium complex or a ruthenium complex or an iron complex. It may comprise a redox complex.

FG may have structure (III), where L′ is a linker group, X′ is S, O or NR^{N}, where R^{N} is H or alkyl, and R′ is selected from the group consisting of H, alkyl, alkoxy or OCR^{c}R^{d} coupled to an atom in L′ so as to form a six-membered ring, wherein R^{c} and R^{d} are independently H or optionally substituted alkyl. In structure (III), X′ may be S. In structure (III), L′ may have structure OCH₂.

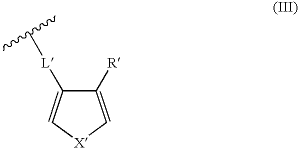

The compound of structure (I) may have structure (IV).

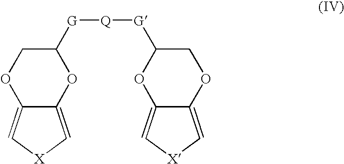

In structure (IV), G and G′ may be the same and X and X′ may be the same.

In an embodiment of the invention there is provided a compound having the general formula below:

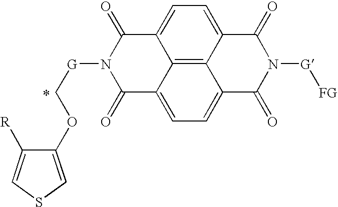

wherein:

each of G and G′ is an independently selected linking moiety comprising 0 to 20 main chain atoms, optionally substituted;

FG is a functional moiety comprising at least one oxygen or nitrogen atom or a transition metal complex;

R is selected from hydrogen, alkyl, alkoxy or —O—CH₂-fused with the carbon marked with a * to form a fused six-membered ring.

There is also provided a polymer comprising monomers of at least one compound of the above embodiment. The polymer may be formed by electropolymerisation.

In another embodiment of the invention there is provided a compound having the general formula below:

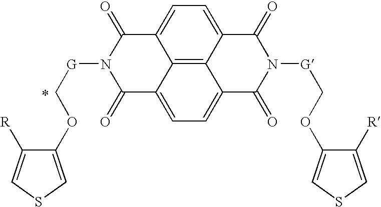

wherein:

each of G and G′ is an independently selected linking moiety comprising 0 to 20 main chain atoms, optionally substituted;

each of R and R′ is selected from hydrogen, alkyl, alkoxy or —O—CH₂-fused with carbon marked with a * to form a fused six-membered ring.

There is also provided a polymer comprising monomer groups derived from at least one compound of the first aspect. The polymer may be formed by electropolymerisation.

In another embodiment of the invention there is provided a compound of structure (IIa), wherein G, G′ and FG are as described above.

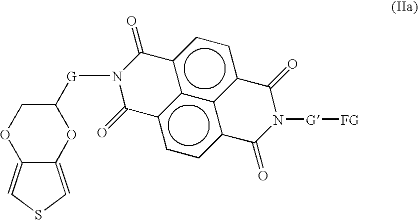

In another embodiment of the invention there is provided a compound of structure (IVa), wherein G and G′ are as described above.

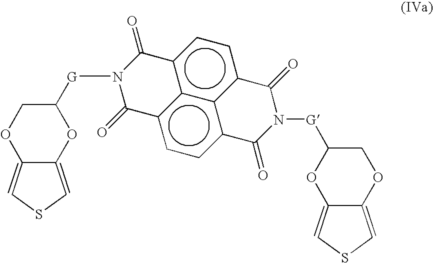

The invention also provides a composition comprising a compound of the first aspect and at least one solvent, diluent, or excipient. The composition may be a solution or it may be a suspension or it may be an emulsion or it may be a microemulsion or it may be a dispersion.

In a second aspect of the invention there is provided an electrically conducting polymer comprising monomer units derived from a compound according to the first aspect of the invention.

The polymer may be a copolymer and may additionally comprise monomer units derived from a second monomer, said second monomer being an optionally substituted thiophene, an optionally substituted pyrrole or an optionally substituted furan, or a mixture of any two or more of these, wherein said optionally substituted thiophene, optionally substituted pyrrole or optionally substituted furan is unsubstituted in the 2 and 5 positions. The second monomer may be a 3,4-ethylenedioxythiophene. The second monomer should be capable of electrocopolymerising with the compound of the first aspect. It may be capable of electrocopolymerising therewith to form a conducting polymer. The polymer may have no monomer units other than those derived from the compound of the first aspect, or may have no monomer units other than those derived from the compound of the first aspect and from the second monomer or it may have additional monomer units provided that the polymer is electrically conducting.

In a third aspect of the invention there is provided a process for making a compound of structure (I) as defined above, said process comprising reacting a compound of structure (V) and a compound of structure FG-G′-NH₂ with a compound of structure A-Q-A, wherein A is a functional group capable of coupling with an amine group and Q is a group capable of binding with dsDNA.

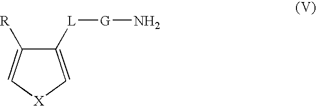

The following options may be used in conjunction with the third aspect, either individually or in any appropriate combination.

A may be an anhydride group.

Q may be a naphthalene group.

The compound of structure A-Q-A may be naphthalene dianhydride.

The compound of structure FG-G′-NH₂ may have structure (V). In this case, the process comprises reacting a compound of structure (V) with naphthalene dianhydride.

The invention also provides a compound of structure (I) as defined above when made by the process of the third aspect.

In a fourth aspect of the invention there is provided a process for making a polymer according to the second aspect, said process comprising electropolymerising a monomer of structure (I) as described above.

In an embodiment the monomer of structure (I) is mixed with a second monomer, said second monomer being an optionally substituted thiophene, an optionally substituted pyrrole or an optionally substituted furan, wherein said optionally substituted thiophene, optionally substituted pyrrole or optionally substituted furan is unsubstituted in the 2 and 5 positions. In this embodiment the process comprises electrocopolymerising the monomer of structure (I) with the second monomer.

In another embodiment there is provided a process for making a polymer according to the second aspect, said process comprising making a monomer of structure (I) using the process of the third aspect of the invention, and electropolymerising said monomer.

In another embodiment there is provided a process for making a polymer according to the second aspect, said process comprising making a monomer of structure (I) using the process of the third aspect of the invention, and electrocopolymerising said monomer with a second monomer, said second monomer being an optionally substituted thiophene, an optionally substituted pyrrole or an optionally substituted furan, wherein said optionally substituted thiophene, optionally substituted pyrrole or optionally substituted furan is unsubstituted in the 2 and 5 positions.

The invention also provides a polymer when made by the process of the fourth aspect.

In a fifth aspect of the invention there is provided a method for determining the presence or absence of a dsDNA in a sample comprising:

exposing the sample to a compound according to the first aspect, or made by the third aspect, or to a polymer according to the second aspect, or made by the fourth aspect,

comparing a signal from the compound or polymer before said exposing to a corresponding signal of the compound or polymer after said exposing, and

determining the presence or absence of a dsDNA in the sample from said comparing.

The signal may be selected from the group comprising absorbance (e.g. absorbance maximum) of UV/visible light, electrical impedance, electrical resistance, electrical conductivity and onset potential for electrical conductivity.

The method may be a method for determining the concentration of dsDNA in the sample.

In an embodiment the method comprises the following steps;
(i) measuring a signal from the compound or polymer;
(ii) mixing the sample with the compound or polymer to form a mixture under conditions facilitating binding of the compound or polymer with dsDNA;
(iii) measuring a signal of said mixture; and
(iv) determining a difference in the signals between (i) and (iii), wherein said difference in the signals is indicative of the presence of said dsDNA.

In a sixth aspect of the invention there is provided a sensor for detecting the presence or absence of dsDNA in a sample, said sensor comprising an electrically conducting polymer according to the second aspect, or made by the fourth aspect.

In a seventh aspect of the invention there is provided a method for determining the presence or absence of a specific polynucleotide sequence in a sample comprising:

providing an electrode having bonded to the surface thereof a polynucleotide sequence complementary to said specific nucleotide sequence;

exposing the electrode to the sample and to a compound according to the first aspect, or made by the third aspect;

supplying a cyclic voltage to the electrode so as to electropolymerise said compound to form a conducting polymer;

measuring a cyclic voltammogram of the conducting polymer on the electrode;

comparing said voltammogram with the voltammogram of a control electrode; and

determining the presence or absence of the specific polynucleotide sequence in the sample from said comparing.

In an eighth aspect of the invention there is provided a method for determining the presence or absence of a specific polynucleotide sequence in a sample comprising:

providing an electrode having bonded to the surface thereof a polynucleotide sequence complementary to said specific nucleotide sequence;

exposing the electrode to the sample;

supplying a cyclic voltage to the electrode in the presence of a compound according to the first aspect, or made by the third aspect, so as to electropolymerise said compound to form a conducting polymer;

measuring a cyclic voltammogram of the conducting polymer on the electrode;

comparing said voltammogram with the voltammogram of a control electrode; and

determining the presence or absence of the specific polynucleotide sequence in the sample from said comparing.

The method of either the seventh or the eighth aspect may be a method for determining a concentration of said specific polynucleotide sequence. In this case the step of comparing comprises comparing the magnitude of a current in said voltammogram with the magnitude of a current in a voltammogram measured using a known concentration of said specific nucleotide sequence, and the step of determining comprises determining the concentration of the specific polynucleotide sequence in the sample from the comparing.

In either the seventh or the eighth aspect the step of supplying the cyclic voltage may be conducted so as to form a conducting polymer whereby groups on said polymer are intercalated with a double stranded polynucleotide, if present, on the electrode.

In a ninth aspect of the invention there is provided a compound of structure (I) according to the first aspect, or made by the process of the third aspect, or a polymer according to the second aspect, or made by the process of the fourth aspect, whereby group Q of said compound or polymer is intercalated with a dsDNA or a double stranded polynucleotide. In an embodiment the dsDNA or double stranded polynucleotide is coupled to, optionally bonded to, an electrode. The electrode may be a gold electrode, or a platinum electrode or a palladium electrode. The dsDNA or double stranded polynucleotide may be coupled to the electrode by means of a sulfur-metal (e.g. sulfur-gold, sulfur-platinum or sulfur-palladium) bond.

In a tenth aspect of the invention there is provided a process for making a polymer according to the second aspect, or made by the process of the fourth aspect, whereby group Q of said polymer is intercalated with a dsDNA or a double stranded polynucleotide, said process comprising electropolymerising a compound of structure (I) according to the first aspect, or made by the process of the third aspect, in the presence of the dsDNA or double stranded polynucleotide. In an embodiment the compound of structure (I) is intercalated with the dsDNA or double stranded polynucleotide during said electropolymerising. In some embodiments the dsDNA or double stranded polynucleotide is coupled to, optionally bonded to, an electrode. The electrode may be a gold electrode, or a platinum electrode or a palladium electrode. The dsDNA or double stranded polynucleotide may be coupled to the electrode by means of a sulfur-metal (e.g. sulfur-gold, sulfur-platinum or sulfur-palladium) bond.

### BRIEF DESCRIPTION OF THE DRAWINGS

A preferred embodiment of the present invention will now be described, by way of example only, with reference to the accompanying drawings wherein:

FIG. 1 shows plots of electropolymerization of (A) bis-EDOT-ND 4a and (B) bis-EDOT-ND 4b at a scan rate of 100 mV/s. Electropolymerization was performed in 0.1 M of nBu₄NPF₆/CH₂Cl₂ solution containing 10 mM of the respective monomers.

FIG. 2 shows plots of electropolymerization of monomer mixtures of bis-EDOT-ND 4b and EDOT at a scan rate of 100 mV/s. The monomer mixture contained of (A) 50% and (B) 10% of 4b. Electropolymerization was performed in 0.1 M of nBu₄NPF₆/CH₂Cl₂ solution containing 10 mM of the monomer mixtures.

FIG. 3 shows UV-visible spectra of poly4b-co-polyEDOT films on ITO electrode. The films were electropolymerized from monomer mixtures containing of (A) 50% and (B) 10% of 4b in 0.1 M of nBu₄NPF₆/CH₂Cl₂ solution. The spectra was normalized based on the absorption peak of polyEDOT (λₘₐₓ=600 nm).

FIG. 4 shows UV-visible absorption spectra of 25-μM bis-EDOT-ND (A) 4a, (B) 4b, and (C) 4c in PBS buffer in the presence of (1) 0, (2) 50, (3) 100, (4) 150 and (5) 200 μM of double-stranded salmon sperm DNA (in base pair). Inset: enlarged UV-visible absorption spectra of the intercalator binding area.

FIG. 5 shows (A) UV-Vis absorption spectra and (B) cyclic voltammograms of EDOT-ND-Os (—), EDOT-ND-EDOT (---), and Os(bpy)₂Cl₂ (...). UV-Visible spectra were measured in ethanol solution. Cyclic voltammograms were measured in 0.1 M nBu₄NPF₆/CH₃CN (EDOT-ND-Os and EDOT-ND-EDOT) and PBS (Os(bpy)₂Cl₂) at a scan rate of 100 mV/s. UV-Vis absorption spectra of 25 μM of (C) EDOT-ND-Os and (D) EDOT-ND-EDOT in PBS buffer solution containing 0, 25, 50, and 75 μM (from top to bottom) of salmon sperm DNA. The concentration of DNA is based on base pairs.

FIG. 6 shows (A) Square wave voltammograms of EDOT-ND-Os bound to DNA capture probe hybridized with 20 pM complementary target (—) 100 pM non-complementary target (---), and no target (...). (B) Amperomatric signal from biosensor electrodes hybridizing (hollow) 100 pM complementary target, (gray) 20 pM complementary target, and (black) 100 pM non-complementary target from (A) at 0.14 V after signal substraction of blank experiment (no target). (C) Cyclic voltammograms of PEDOTs formed on assembled biosensor electrodes as described in (A) after seed-mediated electropolymerization of 5 mM EDOT-OH. (D) Amperomatric signal from biosensor electrodes hybridizing (hollow) 100 pM complementary target, (gray) 20 pM complementary target, and (black) 100 pM non-complementary target from (C) at 0.3 V (oxidation) after signal substraction of blank experiment (no target). The voltammograms were measured in aqueous solution containing 0.1 M LiClO₄ as supporting electrolyte.

FIG. 7 shows NMR spectra of selected compounds from the examples.

FIG. 8 shows a scheme illustrating electrochemical DNA detection using an EDOT-grafted intercalator.

### DEFINITIONS

As used herein the term “intercalation” refers to the process by which an entity, reversibly or irreversibly, is included between two or more other entities. The entities may be whole molecules, parts or functional groups thereof.

As used herein the term “biosensing” refers to the detection of an analyte that combines with a biological component via a physicochemical means.

As used herein the term “alkyl” includes within its meaning monovalent, saturated, straight and branched chain and cyclic hydrocarbon radicals.

As used herein the term “alkoxy” includes within its meaning any alkyl group linked to an oxygen.

### DETAILED DESCRIPTION OF THE INVENTION

The invention provides compounds of structure (I). These compounds may be capable of intercalating double stranded polynucleotides. They may be capable of selectively binding to double stranded polynucleotides.

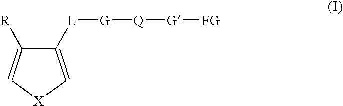

In structure (I), as well as in structures (II), (IIa), (III), (IV), (IVa) and (V), the following descriptions of the various parts (if present) apply:

X and X′ may, independently, be S, O or NR^{N}, where R^{N} is H or alkyl. In some embodiments at least one of X and X′ is S. In further embodiments both X and X′ are S.

L and L′ are linker groups. They may be the same or they may be different. They may, independently, be OCH₂, OCR^{x}R^{y} (where R^{x} and R^{y} are independently selected from the group consisting of H and an alkyl group) or OCH (in which the carbon atom is bonded to R or to R′: see below).

Q is a group capable of binding with dsDNA. It may be capable of intercalating dsDNA. It may be capable of binding dsDNA by a threading intercalation mode. It may be capable of complexing with dsDNA. It may be capable of selectively binding or complexing with dsDNA. It may be a naphthalene diimide group. The naphthalene diimide group may be unsubstituted. It may be substituted. It may be substituted with one or more (e.g. 2, 3 or 4) alkyl or aryl groups. Intercalation may be considered to be the reversible inclusion or insertion of a molecule or a group on a molecule between two other molecules or groups.

G and G′ are, independently, absent or have between 1 and 30 main chain atoms and, if present, are optionally substituted. G and G′ may be the same. They may be different. They may be both absent, or one or both may be present. In the event that G is absent, the group -L-G-Q- is -L-Q-. Similarly, in the event that G′ is absent, the group -Q-G′-FG is -G-FG. G and G′, if present, may, independently, have 1 to 30 main chain atoms, or may have 1 to 20, 1 to 12, 1 to 6, 1 to 4, 6 to 30, 12 to 30, 20 to 30, 6 to 20, 12 to 20 or 6 to 12 main chain atoms, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 main chain atoms. The main chain atoms may all be carbon, or some may be carbon and some may be oxygen. In some embodiments, some of the main chain atoms in G and G′ are nitrogen. Suitable examples of G and G′ include CH₂, (CH₂)₂, (CH₂)₃, CH₂O(CH₂)₆, CH₂OCH₂(CH₂OCH₂)₂CH₂, CH₂OCH₂(CH₂OCH₂)₄CH₂ and CH₂OCH₂(CH₂OCH₂)₃CH₂. G and G′, independently, may comprise one or more (e.g. 1, 2, 3, 4, 5 or 6) polyether groups.

FG is a functional moiety comprising at least one O or N atom or a transition metal complex. FG may be capable of enhancing the binding of the compound of structure (I) or a polymer or copolymer thereof, with a double stranded polynucleotide. It may be an electrically neutral group. It may be a positively charged group. Examples of FG include OH, NH₂, imidazolyl, pyridinyl or metal complexes, e.g. inorganic complexes based on redox couples. Suitable redox couples and complexes include Fe²⁺/Fe³⁺, Os²⁺/Os³⁺, Ru²⁺/Ru³⁺, Os(bipyridine)₂Cl(imidazole), Os(bipyridine)₂Cl(pyridine) Ru(bipyridine)₂Cl(imidazole), Ru(bipyridine)₂Cl(pyridine), ferrocene etc. FG may alternatively have structure (III). In this latter instance, the compound of structure (I) has two electropolymerisable groups. These may be the same, or they may be different. FG may be capable of electrostatically binding to dsDNA. It may be capable of enhancing the binding (or intercalation) of group Q with dsDNA. The enhancement may be due to electrostatic binding. Thus in some embodiments when -Q-G′-FG binds to dsDNA, Q intercalates with the dsDNA (in particular with the hydrophobic interior region of the dsDNA) and FG binds electrostatically to a hydrophilic region of the dsDNA. An example of a compound in which FG is (III) is (IV), in particular (IVa).

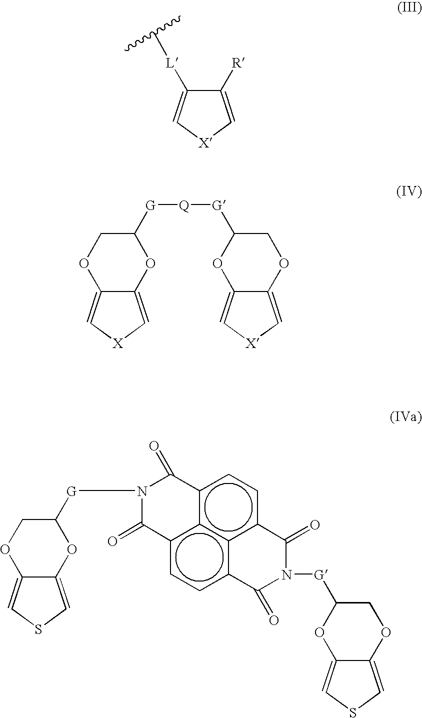

In many cases the compound of structure (I) will be asymmetric. An example is shown below:

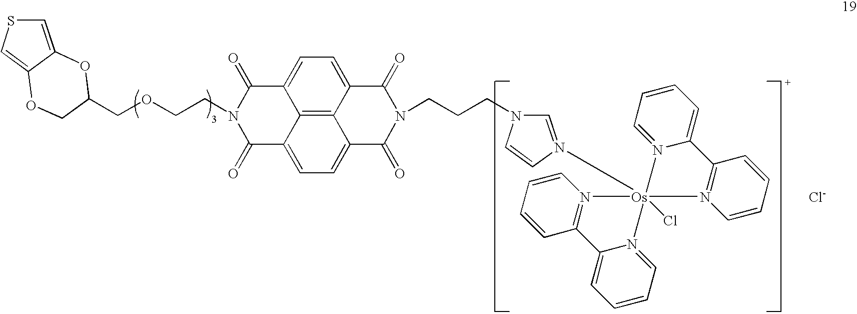

R and R′ are, independently, selected from the group consisting of H, alkyl, alkoxy or OCR^{a}R^{b} coupled to an atom in L or L′ respectively so as to form a six-membered ring, wherein R^{a} and R^{b} are independently H or optionally substituted alkyl. Thus in some embodiments R is OCH₂ coupled to an atom in L so as to form a six-membered ring and/or R′ is OCH₂ coupled to an atom in L′ so as to form a six-membered ring. In particular, one or both of the rings may form part of a 3,4-ethylenedioxythiophene fused ring system.

In the present specification, reference is made to alkyl groups (for example R^{N}, Ra, R^{b}, R^{c}, R^{d}, R^{x} and R^{y} above). In each case, independently, the alkyl group may be selected from the group consisting of:

Linear alkyl groups—these may have between 1 and 20 carbon atoms, or 1 to 12, 1 to 6, 1 to 4, 6 to 20, 12 to 20 or 6 to 12 carbon atoms, e.g. 1, 2, 3, 4, 5, 6, 7, 8, 9, 10, 12, 14, 16, 18 or 20 carbon atoms. Examples include methyl, ethyl, propyl, butyl, hexyl, decyl, dodecyl, octadecyl.

Branched alkyl groups—these may have between 3 and 20 carbon atoms, or 3 to 12, 3 to 6, 60 to 12 or 12 to 20 carbon atoms, e.g. 3, 4, 5, 6, 7, 8, 9, 10, 12, 14, 16, 18 or 20 carbon atoms. They may have 1, 2, 3, 4 or more than 4 branches. Examples include isopropyl, isobutyl, tert-butyl, neopentyl, isopentyl, isooctyl.

Cyclic alkyl groups—these may have between 3 and 20 carbon atoms, or 3 to 12, 3 to 6, 6 to 12 or 12 to 20 carbon atoms, e.g. 3, 4, 5, 6, 7, 8, 9, 10, 12, 14, 16, 18 or 20 carbon atoms. They may be monocyclic, bicyclic or may have 3 or more rings. These may be fused or linked or spiro connected. Examples include cyclopropyl, cyclobutyl, cyclopentyl, cyclohexyl, cyclooctyl, bornyl, adamantyl.

Combinations—the alkyl group may comprise more than one of the above structures. For example it may comprise an alkyl substituted cycloalkyl group or a cycloalkyl substituted alkyl group.

In the present specification, reference is made to aryl groups. These may be monocyclic, bicyclic or polycyclic. They may be fused aromatics. They may be coupled aromatics. They may have 6 to 20 carbon atoms, or 6 to 12, 6 to 8, 8 to 20, 12 to 20 or 8 to 12 carbon atoms, e.g. 6, 8, 10, 12, 14, 16, 18 or 20 carbon atoms. Examples include phenyl, naphthyl, anthracyl, phenylphenyl. The aryl group may be substituted with an alkyl group. Examples include tolyl, ethyl benzyl, ethyl anthracyl etc.

In any or all of the above cases, the alkyl or aryl group may optionally be substituted. The substituent may be an aryl group, a halogen (e.g. F, Cl, or Br) or some other group. Alternatively the alkyl or aryl group may be unsubstituted.

The invention also provides a composition comprising a compound of the first aspect in combination with at least one solvent, diluent, or excipient. The composition may be a solution or a suspension or an emulsion or a microemulsion or a dispersion. The solvent or diluent or excipient may be an aqueous solvent. It may be, or comprise, water. It may be, or comprise, an organic solvent. It may be, or comprise, an alcoholic solvent.

The invention also provides an electrically conducting polymer comprising monomer units derived from a compound according to the first aspect of the invention. In this context, “derived from” need not necessarily indicate that the process for making the polymer involves use of the compound according to the first aspect. Thus a polymer comprising monomer units “derived from (I)” may have monomer units of structure (VI), regardless of how it is formed.

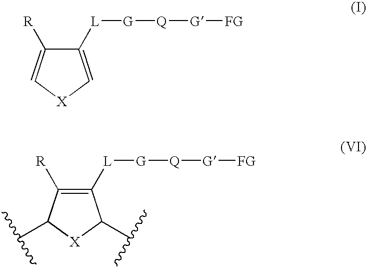

In the event that FG comprises an electropolymerisable group (e.g. in structure (IV)), it will be understood that FG may be incorporated into the polymer backbone also.

The polymer may be a copolymer. It may be a block copolymer, an alternating copolymer, a random copolymer or some other type of copolymer. It may have a structure in which the polymer backbone comprises monomer units derived from two different monomers. The polymer may additionally comprise monomer units derived from a second monomer. The second monomer may be an optionally substituted thiophene, an optionally substituted pyrrole or an optionally substituted furan, or a mixture of any two or more of these. The optionally substituted thiophene, optionally substituted pyrrole or optionally substituted furan may be unsubstituted in the 2 and 5 positions. The second monomer may be a 3,4-ethylenedioxythiophene. The polymerization of the above monomer units may be through the 2 and 5 positions of a 5-membered heterocyclic ring in said monomer units. The polymer may additionally comprise monomer units derived from a third, optionally also fourth, optionally fifth monomer. Each of the third, fourth and fifth monomers may each be, independently, as described for the second monomer unit. The polymer may be a linear polymer. It may be a crosslinked copolymer. It may be doped in order to render it conductive. It may be undoped.

The polymer (or copolymer) may be capable of binding with dsDNA. It may be capable of intercalating dsDNA. It may be capable of binding dsDNA by a threading intercalation mode. It may be capable of complexing with dsDNA. It may be capable of selectively binding or complexing with dsDNA. It may be a naphthalene diimide group.

The polymer may have a molecular weight (Mn or Mw) between about 2,000 and about 2,000,000, or between about 2000 and 1000000, 2000 and 500000, 2000 and 100000, 2000 and 50000, 2000 and 10000, 2000 and 5000, 10000 and 2000000, 100000 and 2000000, 1000000 and 2000000, 10000 and 1000000, 10000 and 100000 or 100000 and 1000000, for example about 2000, 3000, 4000, 5000, 6000, 7000, 8000, 9000, 10000, 15000, 20000, 25000, 30000, 35000, 40000, 45000, 50000, 60000, 70000, 80000, 90000, 100000, 200000, 300000, 400000, 500000, 600000, 700000, 800000, 900000, 1000000, 1500000 or 2000000, or some other suitable molecular weight. It may have a degree of polymerisation of between about 10 and about 10000, or between about 10 and 1000, 10 and 500, 10 and 200, 10 and 100, 10 and 50, 10 and 20, 20 and 10000, 50 and 10000, 100 and 10000, 1000 and 10000, 5000 and 10000, 50 and 5000, 50 and 1000, 50 and 500, 50 and 100, 100 and 1000, 100 and 500 or 500 and 1000, e.g. about 10, 15, 20, 25, 30, 35, 40, 45, 50, 60, 70, 80, 90, 100, 150, 200, 250, 300, 350, 400, 450, 500, 600, 700, 800, 900, 1000, 2000, 3000, 4000, 5000, 6000, 7000, 8000, 9000 or 10000. It may have a narrow molecular weight distribution or it may have a broad molecular weight distribution. It may have a polydispersity of between about 1 and about 10, or between about 1 and 5, 1 and 2, 2 and 10, 50 and 10, 1.5 and 5 or 2 and 5, e.g. about 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 or 10, or may be more than 10.

The polymer (or copolymer) may have a conductivity of at least about 10⁻³ Sm⁻¹, or at least about 5*10⁻³, 10⁻², 5*10⁻², 0.1, 0.5, 1, 5, 10, 50, 100, 200, 500 or 1000 Sm⁻¹, or about 0.001 to 1000, 0.001 to 100, 0.001 to 10, 0.001 to 1, 0.001 to 0.01, 0.01 to 1000, 1 to 1000, 100 to 1000, 0.1 to 100, 0.1 to 10, 0.1 to 1 or 1 to 100 Sm⁻¹, e.g. about 0.001, 0.002, 0.005, 0.01, 0.02, 0.05, 0.1, 0.2, 0.5, 1, 2, 5, 10, 20, 50, 100, 200, 500 or 1000 Sm⁻¹.

The compound of structure (I) may be made by reacting a compound of structure (V) and a compound of structure FG-G′-NH₂ with a compound of structure A-Q-A, wherein A is a functional group capable of coupling with an amine group and Q is a group capable of binding with dsDNA.

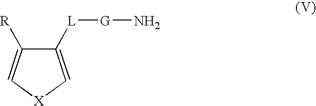

A may be an anhydride group, for example a cyclic anhydride. In this case, an amine can react with A to form an amide (for an acyclic anhydride) or an imide (for a cyclic anhydride). Other groups that can react with amines include acid chlorides (to form amides), N-hydroxysuccinimido esters (to form amides), carbonyl groups (to form imines), alkyl halides (to form amines) alkyl tosylates (to form amines) etc. Q may be as described earlier.

The compound of structure A-Q-A may be naphthalene dianhydride, in which case reaction with amines provides a substituted naphthalene diimide.

In some embodiments of the invention the compound of structure FG-G′-NH₂ has structure (V). In this case, the process comprises reacting a compound of structure (V) with naphthalene dianhydride in order to provide a symmetrically substituted naphthalene diimide. In cases where (V) and FG-G′-NH₂ are not the same, it is possible that more than one bisadduct will form. In this case, the process may comprise a separation step for separating the desired bisadduct (I) from other compounds produced in the reaction.

The reaction of A-Q-A with (V) and FG-G′-NH₂ may be conducted in the presence of a base. Suitably it may be conducted in pyridine, which may function as a base and as a solvent. A catalyst such as zinc acetate may also be added. Typical conditions involve heating the reagents in the solvent, optionally with added base if necessary, for sufficient time (e.g. overnight) to obtain satisfactory conversion to product. A suitable procedure for obtaining the amine reagent (V) is from the corresponding alcohol. The conversion scheme should be such as to not affect the heterocyclic ring of (V). A suitable scheme (exemplified in Scheme 1) starts from the alcohol. This may be esterified with mesyl chloride in the presence of a base such as a trialkyl amine to generate a mesylate ester. This may then be reacted with sodium azide to generate the corresponding azide substituted species. This reaction is commonly conducted in a polar solvent (which may comprise water and/or an alcohol) so as to at least partially dissolve the mesylate ester and the sodium azide. Other reactions which provide activated alcohol derivatives which may be reacted with azide to generate the azide include formation of a tosylate by reaction of the alcohol with sodium hydride and then treatment with a tosylate ester (e.g. tetraethylenglycol ditosylate), followed by reaction of the resulting tosylate with sodium iodide to form the corresponding iodide. This may then be reacted with sodium azide to form the corresponding azide. Reaction of the azide with triphenyl phosphine and base (e.g. hydroxide) provides the corresponding amine.

The compound of structure (I) may be polymerized to generate a polymer according to the second aspect. A suitable process for conducting the polymerisation comprises electropolymerising the monomer of structure (I). This leads to polymerization through the 2 and 5 positions of the heterocyclic ring of (I). The polymerization may be an oxidative polymerization. It may be an oxidative electropolymerisation.

In a suitable electropolymerisation process, the monomer is dissolved in a solution of a supporting electrolyte in an aprotic organic solvent. Suitable supporting electrolytes include tetralkylammonium salts such as tetrabutylammonium hexafluorophosphate. Suitable organic solvents include chloroform, methylene chloride, acetonitrile etc. The solvent should be capable of dissolving the monomer, or monomers, in the concentration required in the electropolymerization reaction. The concentration of electrolyte in solvent may be about 0.02 to about 0.2M, or 0.02 to 0.1, 0.02 to 0.05, 0.05 to 0.2, 0.1 to 0.2 or 0.05 to 0.15M, e.g. about 0.02, 0.03, 0.04, 0.05, 0.06, 0.07, 0.08, 0.09, 0.1, 0.11, 0.12, 0.13, 0.14, 0.15, 0.16, 0.17, 0.18, 0.19 or 0.2M. The solution additionally contains the monomer or monomers (in the case of a copolymerization). The concentration of each monomer, or of the total monomers, may independently be between about 0.1 and 10 mM, or about 0.5 to 10, 1 to 10, 2 to 10, 5 to 10, 0.1 to 5, 0.1 to 2, 0.1 to 1, 0.1 to 0.5, 0.5 to 5, 1 to 5 or 0.5 to 2 mM, e.g. about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9 or 10 mM. Two electrodes are at least partially immersed in the solution, and the electrode potential swept between an upper and a lower potential. The scan rate of the sweeping may be between about 50 and about 500 mV/s or about 50 to 200, 50 to 100, 100 to 500, 200 to 500 or 100 to 200 mV/s, e.g. about 50, 100, 150, 200, 250, 300, 350, 400, 450 or 500 mV/s. The difference between the upper and lower potential may be between about 1 and about 2V or about 1 to 1.5, 1.5 to 2, 1.3 to 1.8, 1.5 to 1.7 or 1.5 to 1.6V, e.g. about 1, 1.1, 1.2, 1.3, 1.4, 1.5, 1.6, 1.7, 1.8, 1.9 or 2V. The upper potential may be between about +1 and about +1.5V versus Ag/Ag⁺, or about +1 to +1.3, +1.2 to +1.5 or +1.2 to +1.4V versus Ag/Ag⁺, e.g. about +1, 1.1, 1.2, 1.3, 1.4 or 1.5V versus Ag/Ag⁺. The lower potential may be between about −0.1 and about −0.5V versus Ag/Ag⁺, or about −0.1 to −0.3, −0.2 to −0.5 or −0.2 to −0.3V versus Ag/Ag⁺, e.g. about −0.1, −0.2, −0.3, −0.4 or −0.5V versus Ag/Ag⁺.

The monomer of structure (I) may be mixed with a second monomer and optionally a third, fourth and/or fifth monomer, prior to electropolymerisation so as to generate a copolymer. The additional monomer(s) may be any suitable monomer capable of copolymerizing with the monomer of structure (I). This may be an optionally substituted thiophene, an optionally substituted pyrrole or an optionally substituted furan. The optionally substituted thiophene, optionally substituted pyrrole or optionally substituted furan may be unsubstituted in the 2 and 5 positions so as to facilitate polymerisation. Suitable comonomers therefore include furan, pyrrole, N-methylpyrrole, thiophene, 3,4-ethylenedioxythiophene, substituted versions of these (preferably unsubstituted at positions 2 and 5) and mixtures of any two or more of the above.

The polymers described above, or the compounds of structure (I), may be used for determining the presence or absence of a dsDNA in a sample. The method comprises exposing the sample to the compound or polymer, and comparing a signal from the compound or polymer before said exposing to a corresponding signal from the compound or polymer after said exposing. Suitable signals that may be used include absorbance (e.g. absorbance maximum) of UV/visible light, electrical impedance, electrical resistance, electrical conductivity and onset potential for electrical conductivity. The method may be a method for determining the concentration of dsDNA in the sample. In this case the method may comprise the step of:
(i) measuring a signal from the compound or polymer;
(ii) mixing the sample with the compound or polymer to form a mixture under conditions facilitating binding of the compound or polymer with dsDNA;
(iii) measuring a signal of said mixture; and
(iv) determining a difference in the signals between (i) and (iii), wherein said difference in the signals is indicative of the concentration of said dsDNA in the sample.

The invention also provides a sensor for detecting the presence or absence of dsDNA in a sample. The sensor comprises an electrically conducting polymer as described herein. The sensor may be incorporated into a detector for detecting the presence or absence of dsDNA in a sample. The sensor may be for example an electrode, whereby at least partial immersion of the sensor into a sample containing dsDNA causes a change in an electrical property of the sensor (e.g. conductivity, resistance, onset potential for conductivity), which may be detected by the detector for detecting the presence of the dsDNA in the sample. The sensor (and the detector) may be capable of determining the concentration of dsDNA in the sample or of providing an output signal which depends on the concentration of dsDNA in the sample. Thus the magnitude of a change of an electrical property of the sensor may be used to determine the concentration of dsDNA in the sample. This may be accomplished by comparing the magnitude of the change with known magnitudes of change for known concentrations of dsDNA.

The monomers described herein (as described in the first aspect, or those made by the third aspect) may be used for determining the presence or absence of a specific polynucleotide sequence in a sample. In this method, an electrode having bonded to the surface thereof a polynucleotide sequence complementary to said specific nucleotide sequence is exposed to the sample and to a compound according to the first aspect, or made by the third aspect. Suitable conditions (temperature, solvent etc.) should be provided so that the complementary nucleotide sequence hybridises with the specific polynucleotide sequence (if present) to form a double strand polynucleotide. The compound can then intercalate into this double strand polynucleotide (if present). The compound has an electropolysable group (e.g. EDOT), and also may contain a group such as a redox group and/or a positively charged group, which assists binding with the polynucleotide. The electrode may optionally be washed in order to reduce non-specific binding of undesirable compounds such as non-complementary sequences. A cyclic voltage is then supplied to the electrode so as to electropolymerise the compound to form a conducting polymer. The presence or absence of conducting polymer is then measured. This may be achieved by measuring a cyclic voltammogram of the conducting polymer on the electrode and comparing the voltammogram with the voltammogram of a control electrode having no conducting polymer. In particular, the current or current variation may be measured. The measurement of the polymer provides greater sensitivity for detecting the polynucleotide sequence relative to enzymatic or electrocatalytic signal amplification, or to an unamplified electrochemical signal.

In a similar method for determining the presence or absence of a specific polynucleotide sequence in a sample, the electrode having bonded to the surface thereof a polynucleotide sequence complementary to said specific nucleotide sequence is exposed to the sample. If the sample contains the specific nucleotide sequence, it will then hybridise with the complementary polynucleotide sequence. The electrode may optionally then be washed in order to reduce non-specific binding of undesirable compounds such as non-complementary sequences. A cyclic voltage to the electrode in the presence of a compound according to the first aspect, or made by the third aspect, so as to electropolymerise said compound to form a conducting polymer. As in the previous method above, a cyclic voltammogram is then measured and compared with a control voltammogram.

Both of the two methods described above may be used for determining a concentration of said specific polynucleotide sequence. In this case the the magnitude of a current, or of a current variation, in the voltammogram is compared with the magnitude of a current, or current variation, in a voltammogram measured using a known concentration of said specific nucleotide sequence. A series of voltammograms may be measured using known polynucleotide concentrations in order to construct a calibration curve for use in determining the concentration in a sample of unknown concentration.

The above methods have described the detection of a specific polynucleotide sequence by use of a compound according to the first aspect of the present invention, or made by the third aspect. It will be clear that other related compounds may be suitable for use in this method, and these are envisaged by the inventors. Thus the compound may be replaced by any compound that 1) has an electropolymerisable group; 2) has a group capable of intercalating a double stranded polynucleotide; and 3) when electropolymerised forms an electrically conducting polymer. Such compounds are themselves also envisaged as aspects of the present invention. The particular compounds included in the first aspect, and made by the third aspect, are merely examples of this general class.

Preferred embodiments of the invention are described below. It is to be understood that the figures and examples provided herein are to exemplify, and not to limit the invention and its various embodiments.

An embodiment of the invention relates to ethylenedioxythiophene (EDOT) monomers coupled with intercalating units for DNA binding. EDOT is selected as the conducting polymer building block due to its high conductivity, low onset potential and excellent stability in aqueous solution after polymerisation. An advantage of the approach of coupling EDOT with intercalating units is the use of conducting polymer as an amplified reporter, whereby, combining this detection scheme with proper device design, the detected amperometric signal could be on the μA to mA level, in contrast with the nA level achieved with voltammetric detection methods. Thus, the detection limit is significantly lowered. Results obtained show significant binding with double-stranded DNA, and formation of homopolymers or copolymers through electropolymerisation.

Provided herein are compositions and methods for improved signal output and reduced detection limit by using conducting polymers based on ethylenedioxythiophene (EDOT) monomers coupled with intercalating units for DNA binding. It will be understood by a person skilled in the art that an advantage of this approach is the use of conducting polymer as amplified reporter. The person skilled in the art will recognize that combining this detection scheme with proper device design, the detected amperometric signal can be on the μA to mA level, in contrast with the nA level achieved with voltammetric detection methods. Thus, the detection limit for DNA is significantly lowered.

Disclosed herein is a synthetic strategy to access threading intercalative EDOT monomers. The EDOT monomers have shown significant binding with dsDNA, and are capable of forming homopolymers or copolymers through electropolymerization. These monomers and polymers are suitable for applications in nucleic acid biosensing.

In accordance with the present invention, compositions, methods and kits are provided for the production and use of conducting polymers based on ethylenedioxythiophene (EDOT) monomers coupled with intercalating units for DNA binding. The methods generally comprise compositions of EDOT polymers to detect nucleic acid.

In one form of the invention the EDOT monomers disclosed herein may be of the following general formulae:

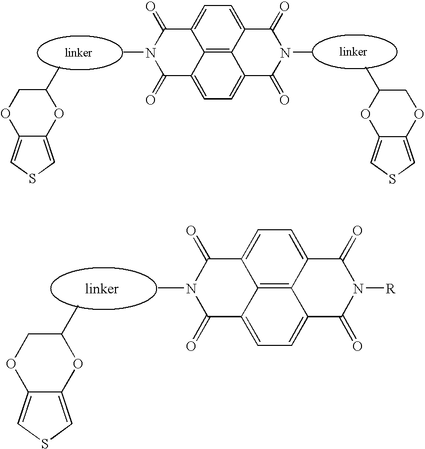

wherein R is a functional moiety comprising at least one oxygen or nitrogen atom or a transition metal complex.

The linker(s) is an independently selected moiety comprising 0 to 20 main chain atoms, optionally substituted. Inorganic complexes coupled with naphthalene diimides (NDs) have been previously synthesized as threading intercalators, and they displayed better selective binding to dsDNA. The inorganic complexes may be based on redox couples of Fe²⁺/Fe³⁺, Os²⁺/Os³⁺, and Ru²⁺/Ru³⁺.

Amino-functionalized EDOT derivatives 3a-c were derived with different linkers as shown in Schemes 1-3. EDOT was selected as the conducting polymer building block due to its high conductivity, low onset potential and excellent stability in aqueous solution after polymerization. Synthesis began with hydroxymethyl-functionalized EDOT (EDOT-OH). In the synthesis of 3b and 3c, hydrophobic and hydrophilic linkers were first introduced onto EDOT-OH through Williamson ether synthesis, followed by nucleophilic substitution to form azide-functionalized EDOT (EDOT-N₃), 2a-c. Azide-functionalized EDOT derivatives 2a-c were subsequently reduced to yield the corresponding amino-functionalized EDOT (EDOT-NH₂), 3a-c.

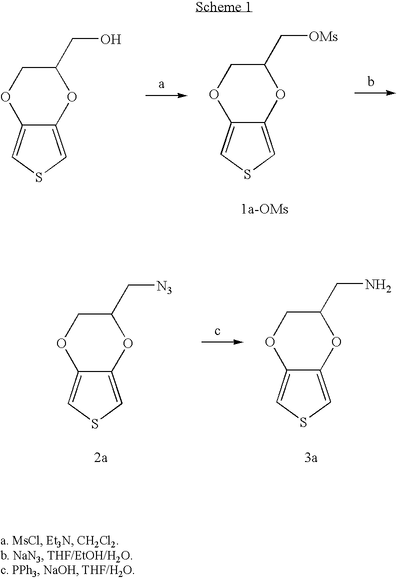

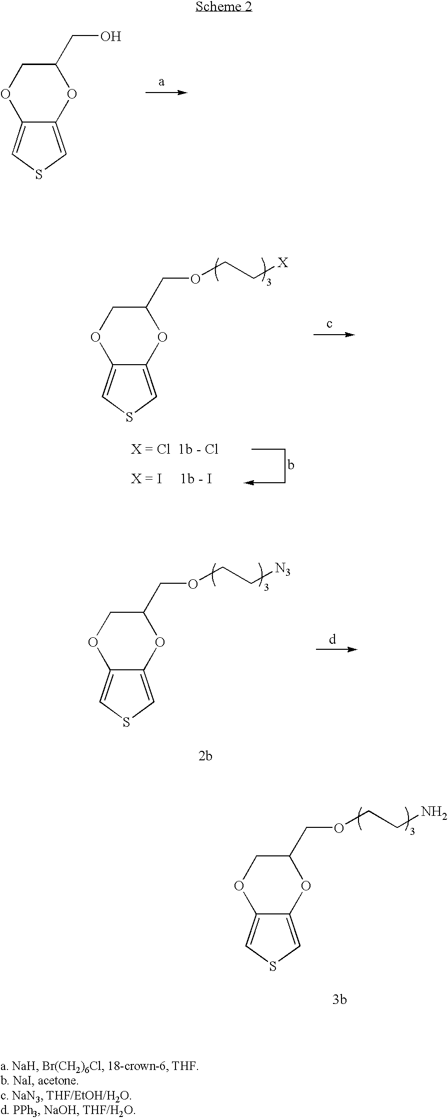

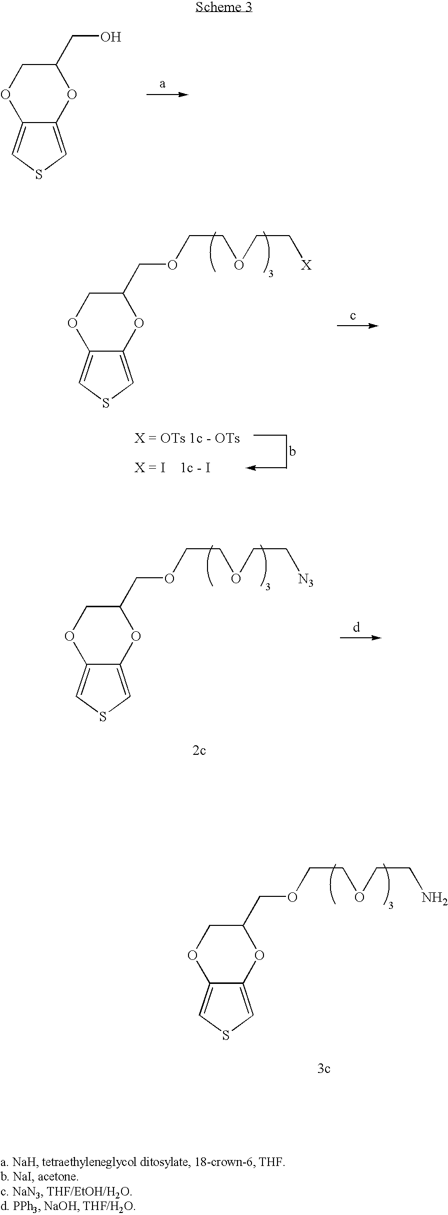

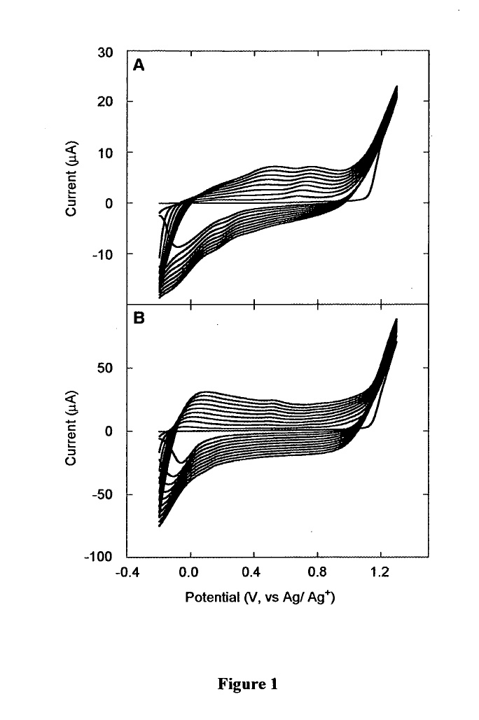

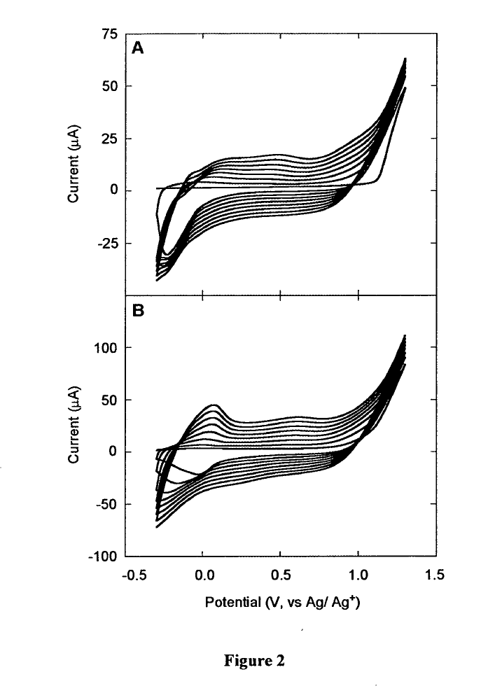

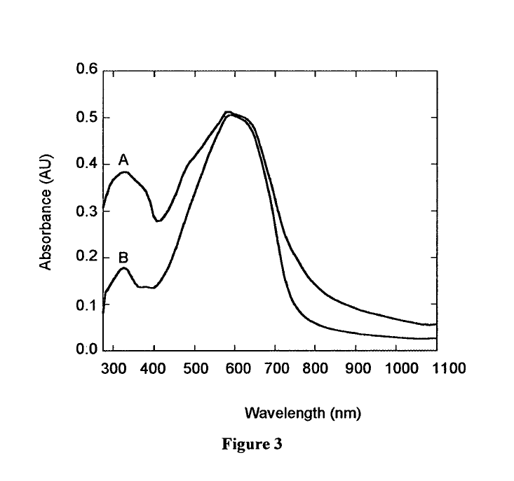

Condensation between 3a-c with 1,4,5,8-naphthalene tetracarboxylic dianhydride provided NDs conjugated to two EDOT moieties (bis-EDOT-ND, 4a-c) in 40-80% yield (Scheme 4). All three bis-EDOT-ND derivatives displayed good solubility in CH₂Cl₂ and dimethyl sulfoxide (DMSO). Compounds 4a and 4b were insoluble in CH₃CN and water, whereas the more hydrophilic 4c showed increased solubility in these solvents. Electropolymerization was successfully performed in CH₂Cl₂ solution containing 10 mM of the bis-EDOT-ND monomers and 0.1 M of tetrabutylammonium hexafluorophosphate (nBu₄NPF₆) as supporting electrolyte by repeated cycling between −0.2 and 1.3 V (referred to as Ag/Ag⁺ reference electrode) at a scan rate of 100 mV/s. Negative shifts in the oxidation current onset after the initial scan and new broad redox waves grew in subsequent scans, indicating polymer growth on the electrode surface (see FIG. 1). Copolymers of these new monomers with other EDOT monomers at different ratios were also electropolymerized under similar conditions (FIG. 2). After normalizing the UV-visible absorption intensity of the polyEDOT backbone (λₘₐₓ=600 nm), a stronger absorption from ND (λₘₐₓ=326 nm) was observed in the copolymer prepared from a monomer mixture containing 50% 4b, compared to the analogous system incorporating only 10% 4b (FIG. 3). This indicated that the composition of the copolymer was directly related to the monomer mixture composition. The successful copolymerization experiments indicated the feasibility of applying polyEDOT as an amplified reporter for nucleic acid biosensing.

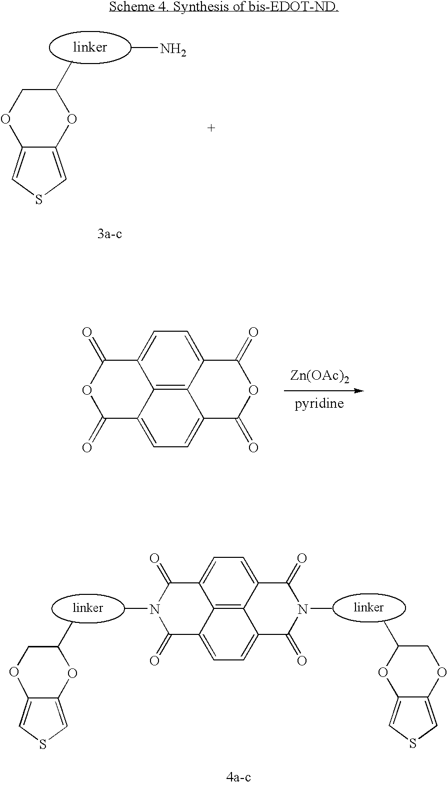

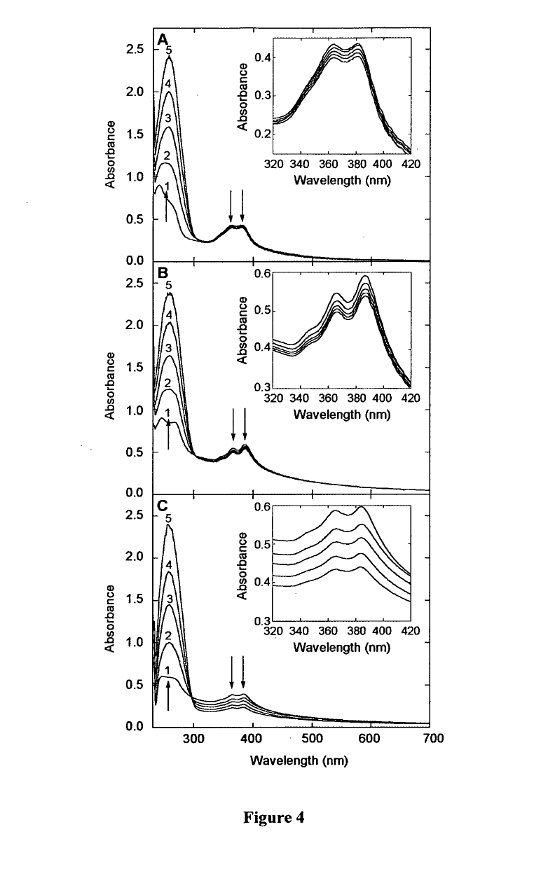

UV-visible spectra of bis-EDOT-ND 4a-c in the presence of increasing amount of double-stranded salmon sperm DNA was first investigated to study the binding between bis-EDOT-NDs with dsDNA. Intercalative binding, where the fused planar aromatic ring system of a threading intercalator is inserted between the base pairs of dsDNA, leads to hypochromism and reduced absorption from ND. Addition of DNA to 4a and 4b at a DNA base pair/bis-EDOT-ND ratio of 8.0 resulted in ˜7% and ˜10% decrease in ND absorption band at 363 and 387 nm (FIG. 4). This limited hypochromism could be attributed to the hydrophobic nature of 4a and 4b. Previously reported ND-based intercalators usually contained charged or metal-centered functional groups linked to ND, allowing better kinetic pathways for ND intercalation into the negatively charged dsDNA. This hypothesis was proven by the much greater 42% absorption reduction when similar experiment was performed on 4c. Bis-EDOT-ND 4c contained a hydrophilic tetraethylene glycol linker. Therefore, it gave rise to better intercalative binding.

A similar synthetic strategy was used to synthesize asymmetric mono-EDOT-ND conjugates 5 (Scheme 5). Condensation of 1,4,5,8-naphthalene tetracarboxylic dianhydride with 1 equivalent of EDOT-NH₂ 3a-c and one equivalent of another functionalized amine yielded the desired product after column chromatography purification. These mono-EDOT-ND derivatives allowed us to introduce charged functional groups (ammonium, pyridinium, imidazolium) or metal-centered redox couples (Fe²⁺/Fe³⁺, Os²⁺/Os³⁺, and Ru²⁺/Ru³⁺). As a result, the intercalative binding properties of EDOT-based intercalators were enhanced.

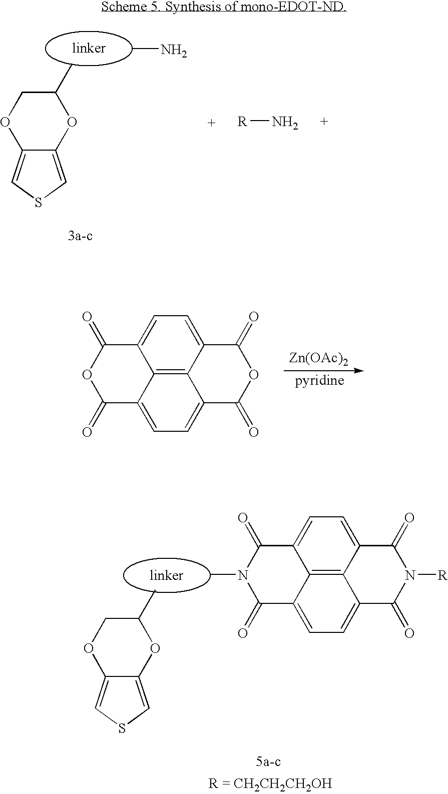

### EXAMPLES

### Example 1

General Methods. Nuclear magnetic resonance (NMR) spectra were recorded on a Bruker Avance 400 spectrometer. Chemical shifts are referenced to residual solvents. High-resolution mass spectra (HR-MS) were recorded on a Finnigan MAT 95XL-T spectrometer. UV-visible spectra were recorded on an Agilent 8453 diode array spectrophotometer. Column chromatography was performed using CombiFlash Companion from Teledyne Isco. Air- and water-sensitive reactions were conducted in an Innovative Technologies glovebox.

Materials. Anhydrous tetrahydrofuran (THF) was purchased from Sigma-Aldrich in a sure-seal bottle. Anhydrous sodium hydride (95%) was purchased from Sigma-Aldrich, and kept and used inside a glovebox. EDOT-OH was prepared following known procedures. Syntheses of 1b-Cl, 1b-I, 1c-OTS, and 1c-I have been reported previously. All other chemicals were of reagent grade and were used as received.

### Example 2

EDOT-OMs (1a-OMs). EDOT-OH (924 mg, 5.4 mmole) was loaded in a 100-mL round-bottom flask with a stir bar, and the flask was backfilled with argon three times. Dry CH₂Cl₂ (15 mL) and triethylamine (0.94 mL, 0.68 g, 6.7 mmole) were introduced, and the reaction mixture was cooled in an ice bath. Methanesulfonyl chloride (0.50 mL, 0.79 g, 6.5 mmole) was added dropwise. The ice bath was removed, and the reaction mixture was stirred for 18 h. Water was added to the mixture, the layers were separated, and the aqueous layer was extracted twice with CH₂Cl₂. The combined organic layers were washed with 5% aqueous H₂SO₄, aqueous saturated NaHCO₃ solution and brine, dried with MgSO₄, and evaporated. The crude product was obtained as a yellow oil, and used directly for the next step.

### Example 3

EDOT-N₃ (2a). Crude product 1a-OMs from the previous step (5.4 mmole) was dissolved in THF (5 mL) and EtOH (10 mL) in a 100-mL round-bottom flask, and freshly prepared aqueous NaN₃ solution (2.80 g, 43.2 mmole of NaN₃ in 10 mL of H₂O) was added. The mixture was stirred under reflux condenser at 80° C. for 48 h. The majority of the organic solvent was removed by a rotary evaporator, then the aqueous layer was extracted 3 times with ethyl acetate. The combined organic layers were dried with MgSO₄ and evaporated. The product was purified on silica gel flash column (hexane/ethyl acetate=20:1). The product 2a (985 mg, 93%) was obtained as a thick colorless oil. ¹H NMR (400 MHz, CDCl₃): δ 6.38 (dd, 2H, J=11.2, 3.6 Hz), 4.33 (ddd, 1H, J=12.0, 6.8, 2.4 Hz), 4.21 (dd, 1H, J=12.0, 2.4 Hz), 4.07 (dd, 1H, J=12.0, 6.8 Hz), 3.59 (dd, 1H, J=13.2, 6.0 Hz), 3.50 (dd, 1H, J=13.2, 6.0 Hz).

### Example 4

EDOT-NH₂ (3a). The azide 2a (1.43 g, 7.2 mmole) was dissolved in THF (25 mL) in a 100-mL round-bottom flask with a stir bar, and triphenylphosphine (PPh₃; 2.08 g, 7.9 mmole) was added as a solid. Vigorous evolution of nitrogen was observed. The reaction was heated at 50° C. for 1 h, whereupon freshly prepared NaOH solution (2 M, 25 mL) was added, and the mixture was heated with vigorous stirring for another 2 h. The majority of THF was removed by a rotary evaporator after acidification with concentrated HCl (pH<3). The aqueous layer was extracted 3 times with CH₂Cl₂, and the combined organic layers were discarded. NaOH was then added to the aqueous layer, and the resulting solution (pH>8) was extracted three times with CH₂Cl₂. The combined organic layers were dried with Na₂SO₄ and evaporated. Product 3a (1.23 g, 100%) was obtained as a colorless viscous liquid. ¹H NMR (400 MHz, CDCl₃): δ 6.33 (dd, 2H, J=8.8, 4.8 Hz), 4.21 (dd, 1H, J=11.2, 2.0 Hz), 4.13 (ddd, 1H, J=11.2, 7.6, 2.0 Hz), 4.07 (dd, 1H, J=12.0, 7.6 Hz), 2.97 (m, 2H), 1.43 (broad s, 2H). ¹³C NMR (100 MHz, CDCl₃): δ 141.7, 141.6, 100.0, 99.6, 75.2, 66.6, 42.3.

### Example 5

EDOT-Cl (1b-Cl). A solution of EDOT-OH (688 mg, 4.00 mmole) and 18-crown-6 (52.8 mg, 0.200 mmole) in anhydrous THF (10 mL) was added dropwise at 0° C. to another air-free flask containing a suspension of sodium hydride (95%, 505 mg, 20.0 mmole) in anhydrous THF (60 mL). The mixture was then introduced to bromochlorohexane (1.60 g, 8.00 mmole) and was refluxed under N₂ overnight. After quenching the excess sodium hydride with water, THF was removed in vacuo. The reaction mixture was then washed with brine, and extracted three times with ethyl acetate. The combined organic phase was dried with MgSO₄, and purified by flash chromatography (hexane/ethyl acetate=9:1) to yield 1b-Cl (680 mg, 2.34 mmole, 59%) as colorless crystals. ¹H NMR (400 MHz, CDCl₃): δ 6.34 (d, 1H, J=4 Hz), 6.32 (d, 1H, J=3.6 Hz), 4.33-4.27 (m, 1H), 4.24 (dd, 1H, J=11.6, 2.4 Hz), 4.06 (dd, 1H, J=11.6, 7.6 Hz), 3.68 (dd, 1H, J=10.4, 5.2 Hz), 3.60 (dd, 1H, J=10.4, 5.6 Hz), 3.53 (t, 2H, 6.8 Hz), 3.50 (t, 2H, J=6.8 Hz), 1.82-1.73 (m, 2H), 1.64-1.55 (m, 2H), 1.50-1.31 (m, 4H).

### Example 6

EDOT-I (1b-I). A solution of 1b-Cl (680 mg, 2.34 mmole) and sodium iodide (1.72 g, 11.5 mmole) was refluxed in acetone (20 mL) for 18 h. The reaction mixture was then filtered to remove precipitates, and acetone was removed in vacuo. It was then dissolved in ethyl acetate, and washed with saturated Na₂S₂O_{3(aq)}. After purification by flash chromatography (hexane/ethyl acetate=9:1), the product was obtained as a viscous, light yellow liquid (647 mg, 1.69 mmole, 74%). ¹H NMR (400 MHz, CDCl₃): δ 6.34 (d, 1H, J=3.6 Hz), 6.32 (d, 1H, J=4 Hz), 4.34-4.27 (m, 1H), 4.24 (dd, 1H, J=11.6, 2 Hz), 4.06 (dd, 1H, J=11.6, 7.6 Hz), 3.68 (dd, 1H, J=10.4, 5.2 Hz), 3.60 (dd, 1H, J=10.4, 5.6 Hz), 3.50 (t, 2H, 6.4 Hz), 3.20 (t, 2H, J=7.2 Hz), 1.80-1.75 (m, 2H), 1.65-1.50 (m, 2H), 1.50-1.31 (m, 4H).

### Example 7

EDOT-N₃ (2b). An aqueous solution (4 mL) of sodium azide (439 mg, 6.76 mmole) was added to a solution of 1b-I (647 mg, 1.69 mmole) in DMF (4 mL). After 18 h of refluxing, DMF was removed by washing with saturated NH₄Cl_{(aq)}. The reaction mixture was extracted in ethyl acetate, the organic layer was washed with water and dried with MgSO₄, and the solvent was removed by a rotary evaporator. After purification by flash chromatography (hexane/ethyl acetate=19:1), 2b was obtained as a viscous light yellow liquid (420 mg, 1.41 mmole, 84%). ¹H NMR (400 MHz, CDCl₃): δ 6.34 (d, 1H, J=3.6 Hz), 6.32 (d, 1H, J=4 Hz), 4.33-4.26 (m, 1H), 4.23 (dd, 1H, J=11.6, 2.4 Hz), 4.05 (dd, 1H, J=11.6, 7.2 Hz), 3.68 (dd, 1H, J=10.4, 4.8 Hz), 3.59 (dd, 1H, J=10.4, 6.4 Hz), 3.49 (t, 2H, 6.4 Hz), 3.26 (t, 2H, J=7.2 Hz), 1.67-1.50 (m, 4H), 1.45-1.30 (m, 4H). ¹³C NMR (100 MHz, CDCl₃): δ 141.6, 141.5, 99.7, 99.6, 77.3, 72.6, 71.8, 69.1, 66.2, 51.4, 29.4, 28.8, 26.5, 25.7. HR-MS (FAB): calcd. for C₁₃H₁₉N₃O₃S+H⁺ 298.1225 [M+H⁺]; found 298.1209.

### Example 8

EDOT-NH₂ (3b). A solution of 2b (297 mg, 1.00 mmole) in THF (5 mL) was mixed with triphenylphosphine (PPh₃, 288 mg, 1.10 mmole), and heated to 50° C. for 1 h. 5 mL of NaOH solution (2 M) were subsequently added, and the reaction was continued for an another 2 h. THF was removed by rotary evaporator, and the aqueous reaction mixture was acidified to pH<3. The aqueous phase was washed with CH₂Cl₂. NaOH was then added, and the resulting solution (pH>10) was extracted with CH₂Cl₂. The organic layer was dried with Na₂SO₄, and the solvent was removed in vacuo. The purified product was distilled with Kugelrohr apparatus (170° C. at 20 mTorr) as a viscous yellow liquid (149 mg, 0.549 mmole, 55%). ¹H NMR (400 MHz, CDCl₃): δ 6.34 (d, 1H J=3.6 Hz), 6.32 (d, 1H, J=4.0 Hz), 4.33-4.26 (m, 1H), 4.24 (dd, 1H, J=11.6, 2.0 Hz), 4.05 (dd, 1H, J=11.6, 7.6 Hz), 3.67 (dd, 1H, J=10.4, 4.8 Hz), 3.59 (dd, 1H, J=10.4, 6.0 Hz), 3.49 (t, 2 H, 6.4 Hz), 2.85 (t, 2H, J=7.2 Hz), 1.67-1.50 (m, 4H), 1.49-1.30 (m, 4H). ¹³C NMR (100 MHz, CDCl₃): δ 151.6, 151.5, 99.7, 99.6, 77.2, 72.6, 71.9, 69.1, 66.2, 53.5, 41.6, 40.5, 32.4, 32.3, 30.1, 29.4, 26.6, 25.8, 25.7. HR-MS (FAB): calcd. for C₁₃H₂₁NO₃S+H⁺ 272.1320 [M+H⁺]; found 272.1321.

### Example 9

EDOT-OTs (1c-OTs). A solution of EDOT-OH (1.72 g, 10.0 mmole) and 18-crown-6 (132 mg, 0.500 mmole) in anhydrous THF (10 mL) was added dropwise at 0° C. into another air-free flask containing a suspension of sodium hydride (95%, 1.26 g, 50.0 mmole) in anhydrous THF (150 mL). The mixture was then added to tetraethylene glycol ditosylate (1.01 g, 20.0 mmole), and was refluxed overnight under N₂. After quenching the excess sodium hydride with water, THF was removed in vacuo. The reaction mixture was then washed with saturated NaCl_{(aq)} and extracted three times with CH₂Cl₂. The combined organic phase was dried with MgSO₄, and purified by flash chromatography (dichloromethane/ethyl acetate=9:1). The product was dried under vacuum to yield a light yellow liquid (1.00 g, 20%). ¹H NMR (400 MHz, CDCl₃): δ 7.80 (d, 1H, J=1.6 Hz), 7.78 (d, 1H, J=1.2 Hz), 6.32 (d, 1H, J=3.6 Hz), 6.31 (d, 1H, J=3.6 Hz), 4.35-4.29 (m, 1H), 4.24 (dd, 1H, J=11.6, 2.0 Hz), 4.15 (t, 2H, J=4.8 Hz), 4.11 (dd, 1H, J=14.4, 7.2 Hz), 4.05 (dd, 1H, J=11.6, 7.6 Hz), 3.76 (dd, 1H, J=10.4, 4.8 Hz), 3.71-3.53 (m, 14H), 2.44 (s, 3H).

### Example 10

EDOT-I (1c-I). A solution of 1c-OTs (1.00 g, 1.99 mmole) and sodium iodide (1.49 g, 9.95 mmole) was refluxed in acetone (20 mL) for 18 h. The reaction mixture was then filtered, and acetone was removed in vacuo. It was then dissolved in CH₂Cl₂, and washed with saturated Na₂S₂O_{5(aq)}. After purification by flash chromatography (dichloromethane/ethyl acetate=19:1), 1c-I (400 mg, 43.6%) was obtained. ¹H NMR (400 MHz, CDCl₃): δ 6.33 (d, 1H, J=3.6 Hz), 6.32 (d, 1H, J=3.6 Hz), 4.36-4.29 (m, 1H), 4.25 (dd, 1H, J=11.6, 2.8 Hz), 4.06 (dd, 1H, J=11.6, 7.2 Hz), 3.77 (dd, 1H, J=9.6, 4.8 Hz), 3.75 (t, 2H, J=5.2 Hz), 3.71-3.63 (m, 13H), 3.26 (t, 2H, J=7.2 Hz).

### Example 11

EDOT-N₃ (2c). A solution of 1c-I (400 mg, 0.873 mmole) in DMF (5 mL) and an aqueous solution (5 mL) of sodium azide (227 mg, 3.49 mmole) were mixed together and refluxed for 18 h. DMF was removed by washing with saturated NH₄Cl_{(aq)}. The reaction mixture was dissolved in CH₂Cl₂, washed with water, and dried with MgSO₄. The crude product was purified by flash chromatography (dichloromethane/ethyl acetate=19:1) to yield a viscous colorless liquid (212 mg, 0.568 mmole, 65%). ¹H NMR (400 MHz, CDCl₃): δ 6.33 (d, 1H, J=4 Hz), 6.32 (d, 1H, J=3.6 Hz), 4.35-4.29 (m, 1H), 4.25 (dd, 1H, J=11.6; 2.4 Hz), 4.06 (dd, 1H, J=11.6, 7.2 Hz), 3.76 (dd, 2H, J=10.8, 5.2 Hz), 3.71-3.52 (m, 15H), 3.39 (t, 2H, J=5.2 Hz). ¹³C NMR (100 MHz, CDCl₃): δ 141.6, 141.5, 99.7, 99.6, 77.3, 72.6, 71.2, 70.7, 70.7, 70.6, 70.5, 70.1, 69.6, 66.1, 50.7. HR-MS (FAB): calcd. for C₁₅H₂₃N₃O₆S+H⁺ 374.1386 [M+H⁺]; found 374.1379.

### Example 12

EDOT-NH₂ (3c). A solution of 2c (100 mg, 0.268 mmole) in THF (3 mL) was mixed with triphenylphosphine (77.3 mg, 0.295 mmole), and was heated to 50° C. for 1 h. 3 mL of NaOH_{(aq)} (2 M) were subsequently added, and the reaction was stirred for another 2 h. THF was removed by rotary evaporator, and the aqueous reaction mixture was acidified to pH<3. The aqueous phase was washed with CH₂Cl₂. NaOH was then added, and the resulting solution (pH>10) was extracted with CH₂Cl₂. The organic layer was dried with Na₂SO₄, and the solvent was removed in vacuo to give a viscous yellow liquid (80.0 mg, 86%). ¹H NMR (400 MHz, CDCl₃): δ 6.29 (d, 1H, J=3.6 Hz), 6.28 (d, 1H, J=3.2 Hz), 4.32-4.25 (m, 1H), 4.21 (dd, 1H, J=11.6, 2 Hz), 4.02 (dd, 1H, J=11.6, 7.6 Hz), 3.72 (dd, 2H, J=10.4, 4.8 Hz), 3.67-3.57 (m, 15H), 3.47 (t, 2H, J=5.2 Hz). ¹³C NMR (100 MHz, CDCl₃): δ 141.5, 141.4, 99.7, 99.6, 72.9, 72.6, 71.1, 70.6, 70.5, 70.5, 70.2, 69.6, 66.1, 53.5, 41.5. HR-MS (FAB): calcd. for C₁₅H₂₅NO₆S+H⁺ 348.1481 [M+H⁺]; found 348.1478.

### Example 13

Bis-EDOT-ND (4a). Naphthalene dianhydride (35.6 mg, 0.133 mmole), EDOT-NH₂ 3a (50.0 mg, 0.292 mmole), and zinc acetate (20.4 mg, 0.093 mmole) were mixed in pyridine (10 mL) and refluxed overnight. The reaction mixture was filtered through a short column of silica gel with CH₂Cl₂ as eluent. The organic solution was then washed with HCl (1 N) and deionized water, dried with MgSO₄, and the solvent was removed by a rotary evaporator. The crude product was further purified by flash chromatography (hexane/ethyl acetate=5:1) to yield 4a as an orange solid (64.0 mg, 0.111 mmole, 78%). ¹H NMR (400 MHz, CDCl₃): δ 8.81 (t, 4H, J=6.4), 6.35 (d, 2H, J=3.6 Hz), 6.29 (d, 2H, J=3.6 Hz), 4.75 (dd, 2H, J=13.6, 7.6 Hz), 4.35 (dd, 2H, J=13.6, 4.8 Hz), 4.26 (dd, 2H, J=12.4, 2.4 Hz), 4.12 (dd, 2H, J=11.6, 6.4 Hz). ¹³C NMR (100 MHz, CDCl₃): δ 162.8, 141.2, 140.9, 131.4, 126.9, 126.5, 100.2, 100.0, 99.9, 71.2, 66.5. HR-MS (FAB): calcd. for C₂₈H₁₈N₂O₈S₂+H⁺ 575.0583 [M+H⁺]; found 574.0575.

### Example 14

Bis-EDOT-ND (4b). Following a similar synthesis procedure as for 4a, 4b was obtained from 3b as an orange solid (52.0 mg, 80%) after flash chromatography (hexane/ethyl acetate=3:1). ¹H NMR (400 MHz, CDCl₃): δ 8.75 (s, 4H), 6.33 (d, 2H, J=3.6 Hz), 6.32 (d, 2H, J=3.6 Hz), 4.32-4.26 (m, 2H), 4.23 (dd, 2H, J=11.6, 2.4 Hz), 4.19 (t, 4H, J=7.6 Hz), 4.05 (dd, 2H, J=11.6, 7.6 Hz), 3.64 (dd, 2H, J=10.4, 4.8 Hz), 6.59 (dd, 2H, J=10.4, 6 Hz), 3.50 (t, 4H, J=3.2 Hz), 1.77-1.64 (m, 4H), 1.61-1.50 (m, 8H), 1.50-1.38 (m, 4H). ¹³C NMR (100 MHz, CDCl₃): δ 162.9, 141.6, 141.5, 131.0, 126.7, 126.6, 99.7, 99.6, 72.6, 71.9, 69.1, 66.2, 40.9, 29.7, 29.4, 28.0, 26.8, 25.8. HR-MS (FAB): calcd. for C₄₀H₄₂N₂O₁₀S₂+H⁺ 775.2359 [M+H⁺]; found 775.2344.

### Example 15

Bis-EDOT-ND (4c). Following a similar synthesis procedure as for 4a, 4c was obtained from 3c as an orange solid (11.0 mg, 30%) after flash chromatography (dichloromethane/ethyl acetate=2:1). ¹H NMR (400 MHz, CDCl₃): δ 8.75 (s, 4H), 6.31 (d, 2H, J=3.6 Hz), 6.29 (d, 2H, J=3.6 Hz), 4.46 (t, 4H, J=6 Hz), 4.34-4.27 (m, 2H), 4.23 (dd, 2H, J=11.6, 2.4 Hz), 4.04 (dd, 2H, J=11.6, 4.8 Hz), 3.84 (t, 4H, J=5.6 Hz), 3.75 (dd, 2H, J=10.4, 4.8 Hz), 3.72-3.64 (m, 10H), 3.64-3.56 (m, 16H). ¹³C NMR (100 MHz, CDCl₃): δ 162.9, 141.5, 141.5, 131.0, 126.7, 126.6, 100.0, 99.7 99.6, 77.9, 72.6, 71.2, 70.6, 70.6, 70.5, 70.1, 69.6, 67.8, 66.1, 39.6. HR-MS (FAB): calcd. for C₄₄H₅₀N₂O₁₆S₂+H⁺ 927.2680 [M+H⁺]; found 927.2698.

### Example 16

Mono-EDOT-ND (5b). A solution of 3b (25.0 mg, 0.092 mmol), naphthalene dianhydride (26.0 mg, 0.092 mmol), aminopropanol (6.91 mg, 0.092 mmol) and zinc acetate (14.1 mg, 0.064 mmol) in pyridine (6 mL) was refluxed overnight. Upon removal of pyridine, the organic solution was then dissolved in CH₂Cl₂, washed with HCl (1 N) and deionized water, and dried with MgSO₄. The solvent was removed by a rotary evaporator. The crude product was further purified by flash chromatography (hexane/ethyl acetate=19:1) to yield a yellow solid (12 mg, 22%). ¹H NMR (400 MHz, CDCl₃): δ 7.78 (t, 4H, J=8 Hz), 6.32 (d, 1H, J=3.6 Hz), 6.31 (d, 1 H, J=3.6 Hz), 4.38 (t, 2H, J=6 Hz), 4.32-4.25 (m, 1H), 4.23 (dd, 1H, J=11.6, 2 Hz), 4.20 (t, 2H, J=7.6 Hz), 4.05 (dd, 1H, J=11.6, 7.2 Hz), 3.67 (dd, 1H, J=10.4, 6 Hz), 3.64 (t, 2H, J=5.6 Hz), 3.59 (10.4, 6 Hz), 3.50 (t, 2H, J=6.8 Hz), 2.02 (q, 2H, J=5.6 Hz), 1.77-1.45 (m, 8H). ¹³C NMR (400 MHz, CDCl₃): δ 163.5, 162.8, 141.6, 141.5, 131.3, 131.0, 126.9, 126.8, 126.7, 126.2, 100.0, 99.7, 99.6, 77.9, 72.6, 71.9, 69.1, 66.2, 59.1, 40.9, 37.5, 30.9, 29.4, 28.0, 26.8, 25.8. HR-MS (FAB): calcd. for C₃₀H₃₀N₂O₈S+H⁺ 579.1801 [M+H⁺]; found 579.1792.

### Example 17

Electrochemistry. All electrochemical measurements were conducted with an Autolab PGSTAT 32 potentiostat (Metrohm) or a CHI830 potentiostat (CH Instruments, Inc.). Cyclic voltammetry was performed in one-chamber and three-electrode cells versus a quasi-internal Ag wire reference electrode (CH Instruments, Inc.) submerged in 0.01 M of AgNO₃/0.1 M of (nBu)₄NPF₆ in anhydrous CH₃CN. Typical cyclic voltammograms (CV) were recorded using platinum button electrodes or indium tin oxide (ITO) coated glass electrodes as the working electrode, and a platinum coil counter electrode.

### Example 18

Electrochemical Polymerization and Copolymerization. For homo-polymerization, 10 mM of monomers 4a-b in 0.1 M of (nBu)₄NPF₆/CH₂Cl₂ solution were oxidatively polymerized when the electrode potential was swept between −0.2 and +1.3 V versus Ag/Ag⁺ at a scan rate of 100 mV/s. For copolymerization, two mixtures with different monomer ratios were prepared in 0.1 M of (nBu)₄NPF₆/CH₂Cl₂. The first mixture contained 1 mM of 4a-b and 9 mM of EDOT, and the other mixture contained 5 mM of 4a-b and 5 mM of EDOT. Oxidative polymerization was done by cyclic voltammetry between −0.3 and +1.3 V versus Ag/Ag⁺ at a scan rate of 100 mV/s.

Several recent reports have demonstrated a greatly amplified signal generated by sandwich DNA assay (capture probe/target DNA/detection probe) through nanoparticle-mediated ((a) Taton, T. A.; Mirkin, C. A.; Letsinger, R. L. Science 2000, 289, 1757. (b) Park, S.-J.; Taton, T. A.; Mirkin, C. A. Science 2002, 295, 1503) or enzyme-catalyzed ((a) Gao, Z. Q.; Rafea, S.; Lim, L. H. Adv. Mat. 2007, 19, 602. (b) Fan, Y.; Chen, X. T.; Trigg, A. D.; Tung, C. H.; Kong, J. M. J. Am. Chem. Soc. 2007, 129, 5437) material growth. Combining this signal amplification method with the advantageous use of intercalators for non-labeling DNA detection, the inventors have found a novel molecular approach of intercalator-mediated polymer growth to achieve sensitive DNA detection based on an ethylenedioxythiophene (EDOT)-grafted intercalator to mediate and promote conducting polymer growth electrochemically on hybridized DNA for signal amplification.

Naphthalenediimide (ND) intercalators conjugated to redox active moieties are applicable to electrochemical DNA detection. On the other hand, conducting polymers derived from EDOT are promising conductive materials due to high conductivity, long-term stability and structure versatility. The inventors have designed two ND-derivatized molecules with EDOT grafted symmetrically (EDOT-ND-EDOT) or asymmetrically (EDOT-ND-Os) via oligoethyleneglycol linkers for improved water solubility.

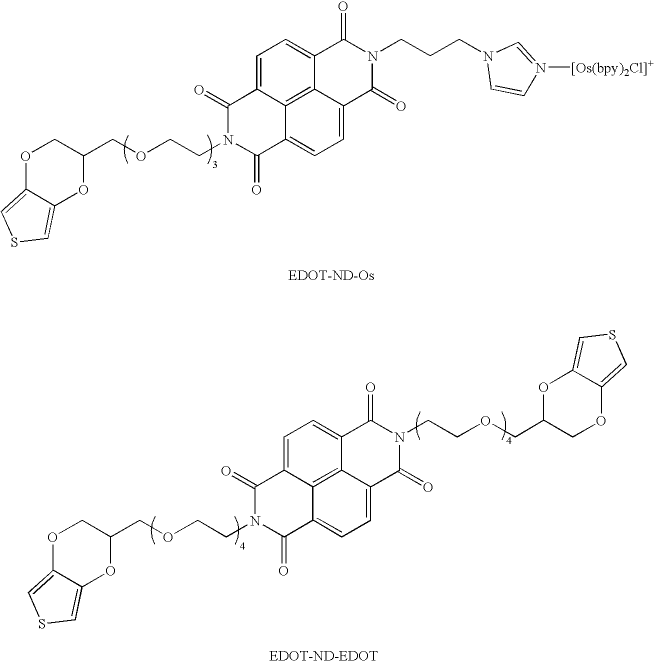

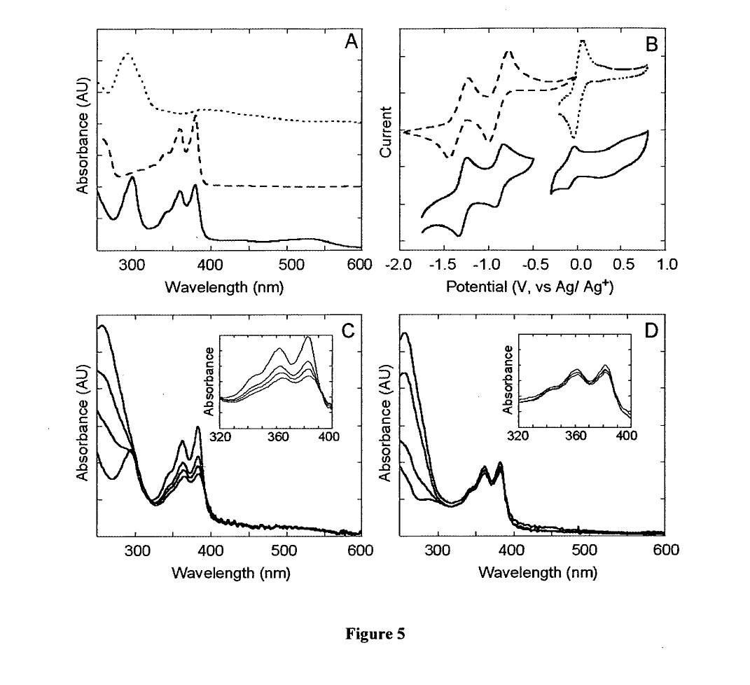

In addition to ¹H and ¹³C NMR and HR-MS, the target compounds were characterized by UV-Vis absorption spectra (FIG. 5A) and cyclic voltammograms (FIG. 5B). UV-Visible spectrum of EDOT-ND-Os exhibits absorption bands from Os(bpy)₂²⁺ (λₘₐₓ=296 nm) and ND (λₘₐₓ=359 and 379 nm) whereas the absorption spectrum of EDOT-ND-EDOT only displays absorption bands arising from ND. Similarly, EDOT-ND-Os displayed redox wave from Os²⁺/Os³⁺ (E^{1/2}=−0.08 V) upon oxidation and redox waves from π-conjugated system of ND (E^{1/2}=−1.30 and −0.88 V) upon reduction.

In intercalation, insertion of the planar aromatic ring between dsDNA base pairs results in hypochromism and red shifts in UV-Vis absorption spectra. As shown in FIG. 5C, addition of salmon sperm DNA to the solution of EDOT-ND-Os at a DNA base-pair/EDOT-ND-Os ratio of 3.0 resulted in 40% decrease in ND absorption bands at 362 and 383 nm. These bands also displayed ˜3 nm red shifts, similar to previous reports on ND with aliphatic tertiary amine side-chains or symmetric Os(bpy)₂²⁺ complex. In contrast, limited hypochromism and red-shifts were observed for EDOT-ND-EDOT, presumably due to limited binding capacity arising from lack of positive charges. For conventional DNA detection, the concentration of target DNA hybrids is so low that intercalation is unlikely to occur by pure diffusion. For positively-charged EDOT-ND-Os, favorable binding occurred between the polyanionic DNA and the cationic intercalator upon additional electrostatic interactions, leading to stronger intercalation behavior. Intercalation properties of EDOT-ND-Os were further studied by competitive displacement assay. When aqueous solution of EDOT-ND-Os was added to dsDNA solution presaturated with thiazole orange (TO), a common fluorescent intercalator, a 50% decrease of fluorescence was observed when the ratio of EDOT-ND-Os/TO reached 4/1. The experiment not only confirmed the intercalation nature of EDOT-ND-Os, but also allowed an estimation of the binding constant of EDOT-ND-Os as 8×10⁴.

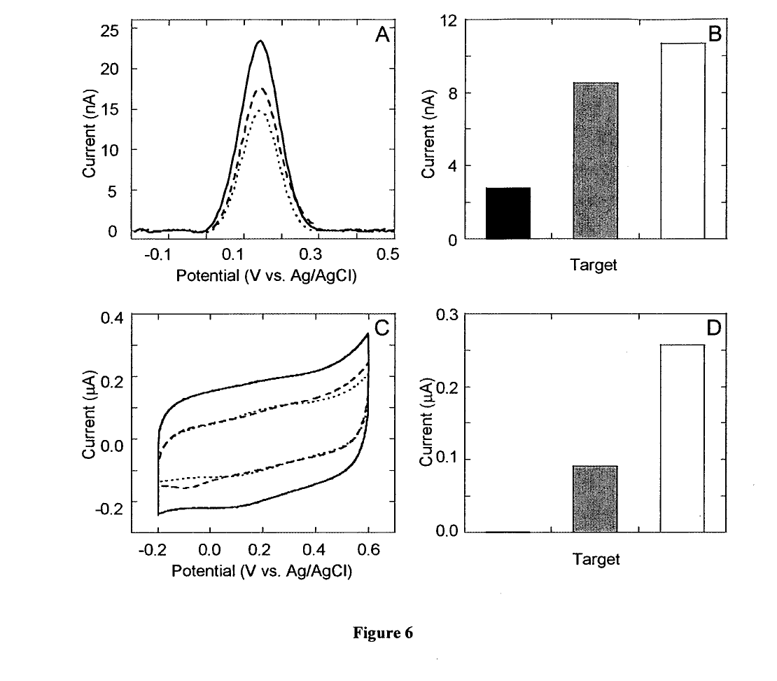

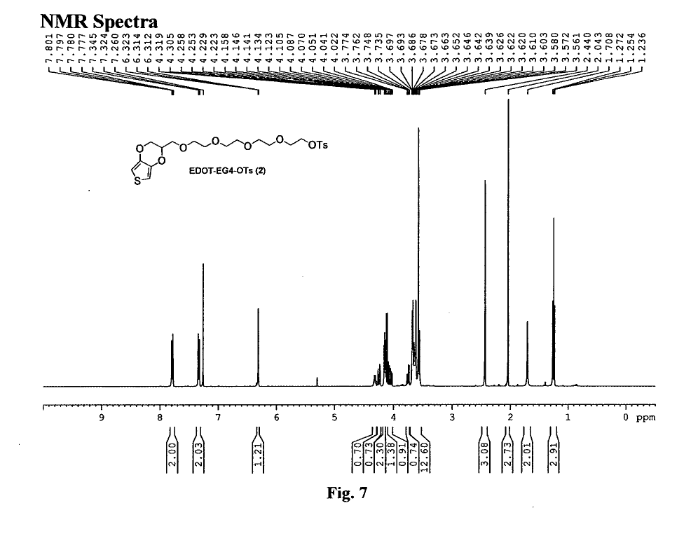

For DNA detection, thiol-functionalized peptide nucleic acid (PNA) capture probe (5′-TTTGAGTCTGTTGCTTGG) and mercaptoundecanol were self-assembled on gold electrode surface. The PNA has a peptide backbone, and the symbols of the structure shown indicate the base, not the backbone. PNA probes were employed to reduce the non-specific binding of cationic EDOT-ND-Os and enhance the hybridization efficiency by reducing the anionic repulsion present when DNA targets hybridize with DNA probes. The probe sequence was designed targeting N1 gene of avian flu virus. After hybridization with the complementary target (5′-CCAAGCAACAGACTCA-AA), EDOT-ND-Os intercalation and washing, redox activity of Os²⁺/Os³⁺ was observed electrochemically (FIG. 6A). Using square-wave voltammetric method, greater amperometric signal was obtained for 20 pM complementary target compared to a control of 100 pM non-complementary target (5′-GGTTCGTTGTCTGAGTTT) and blank experiment without target. However, the signal differences were limited, even after background correction. The peak amperometric output of complementary target was only ˜4 fold of the signal from non-complementary target (FIG. 6B).

The biosensor electrode was then placed in aqueous electrolyte solution (0.1 M LiClO₄) and subjected to cyclic potential between −0.2 and 1.0 V. In the electrode hybridized with complementary target, a greater amperometric difference between the initial and subsequent cycles was obtained due to the formation of oligomers/polymers from surface bound EDOT-ND-Os. To further enhance the signal difference, the electrodes were placed in aqueous electrolyte solution containing 5 mM EDOT-OH. Previous reports on electrochemical growth of conducting polymers have shown that the polymer formed at much lower potential after the initial layer of polymer was deposited (Lima, A.; Schottland, P.; Sadki, S.; Chevrot, C. Synth. Met. 1998, 93, 33). This may be due to the lower over-potential required from improved electron transfer and alternative stepwise polymer propagation steps besides the original radical-radical coupling. After fine-tuning the electrochemical polymer growth condition, it was found that amperometric polymer growth at 0.9 V for 120 seconds was optimal. As shown in FIG. 6C, formation of poly(EDOT-OH) films resulted in larger amperometric output (sub-μA level) in cyclic voltammograms. The voltammograms of control experiment (non-complementary target DNA) and blank experiment (no target) were almost identical, and greater signal difference between complementary and non-complementary targets were observed compared to the electrochemical signal previously observed from Os²⁺/Os³⁺. Applying higher potentials or prolonging the polymerization time resulted in a significant increase of polymer growth on the control experiments, effectively reducing the contrast. After subtraction of the background signal (no target), the contrast of amperometric outputs between complementary and non-complementary DNA targets from the less sensitive cyclic voltammetric method (compared to square-wave method applied in FIG. 6A) was >100 fold.

In conclusion, efficient dsDNA intercalator grafted with monomer unit, EDOT, of conducting polymers was successfully designed and synthesized. The new molecule provides a novel approach to promote electrochemical conducting polymer growth on dsDNA-immobilized electrode for sensitive DNA detection.

### General Experimental for Examples 19 to 21

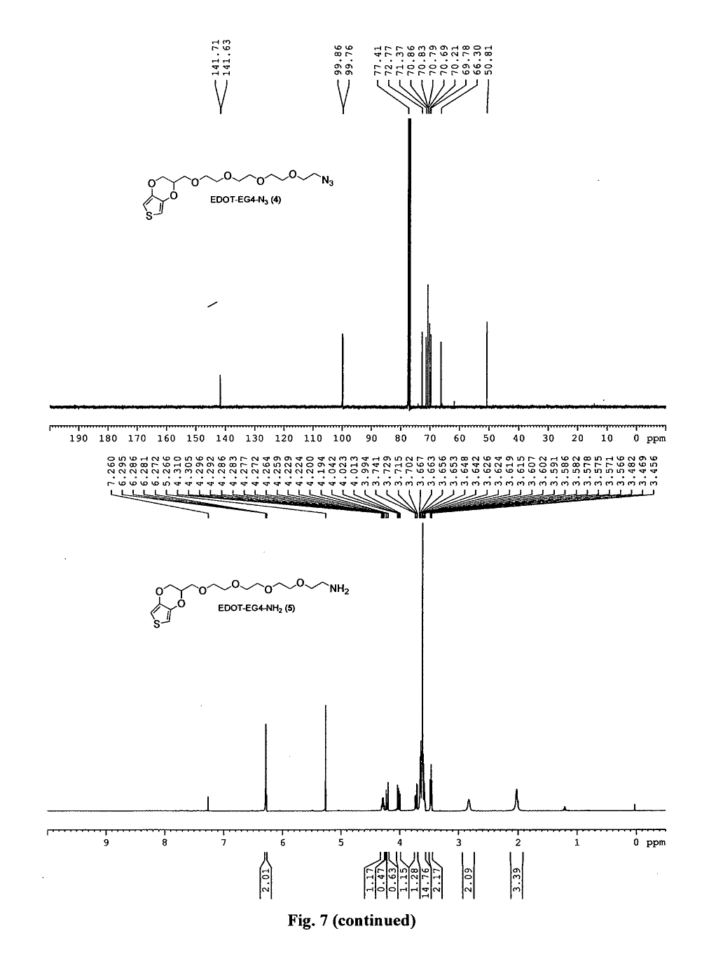

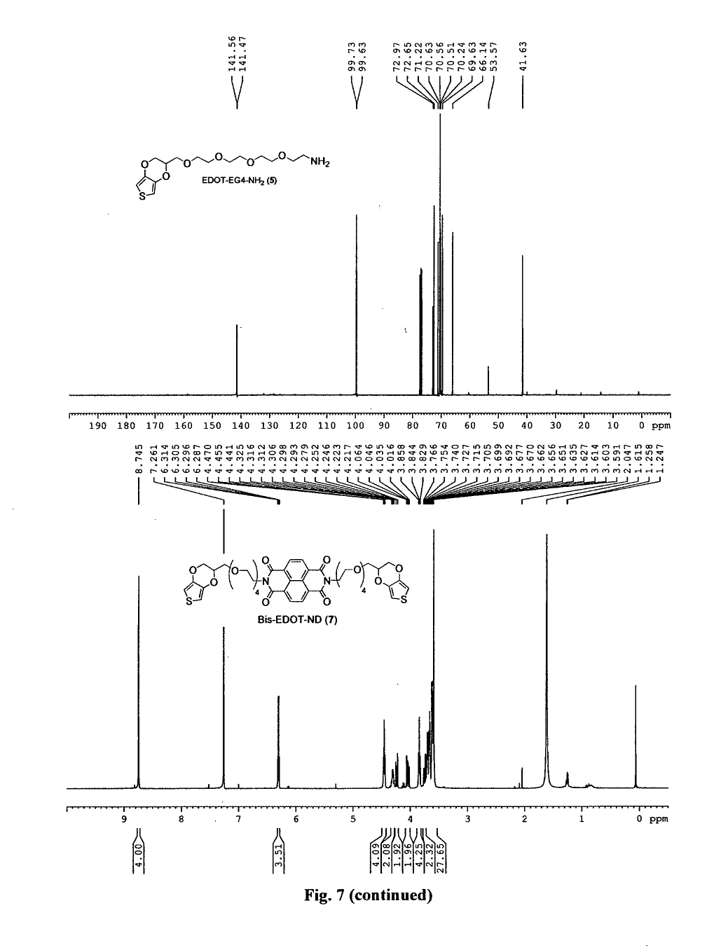

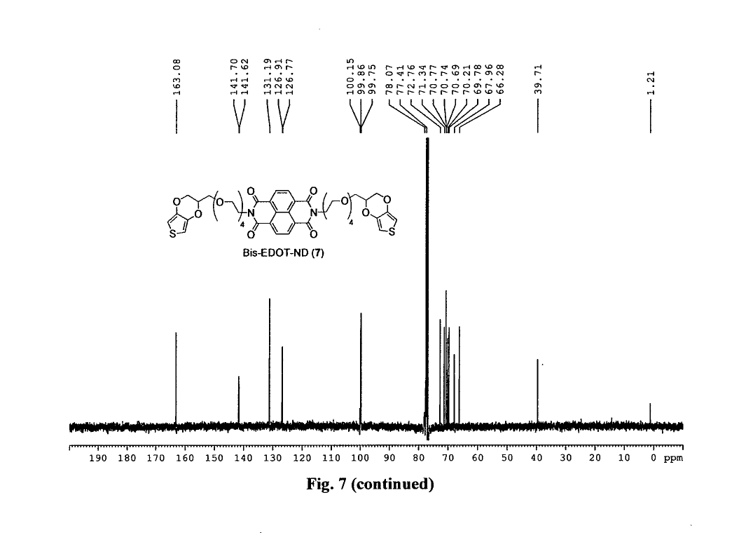

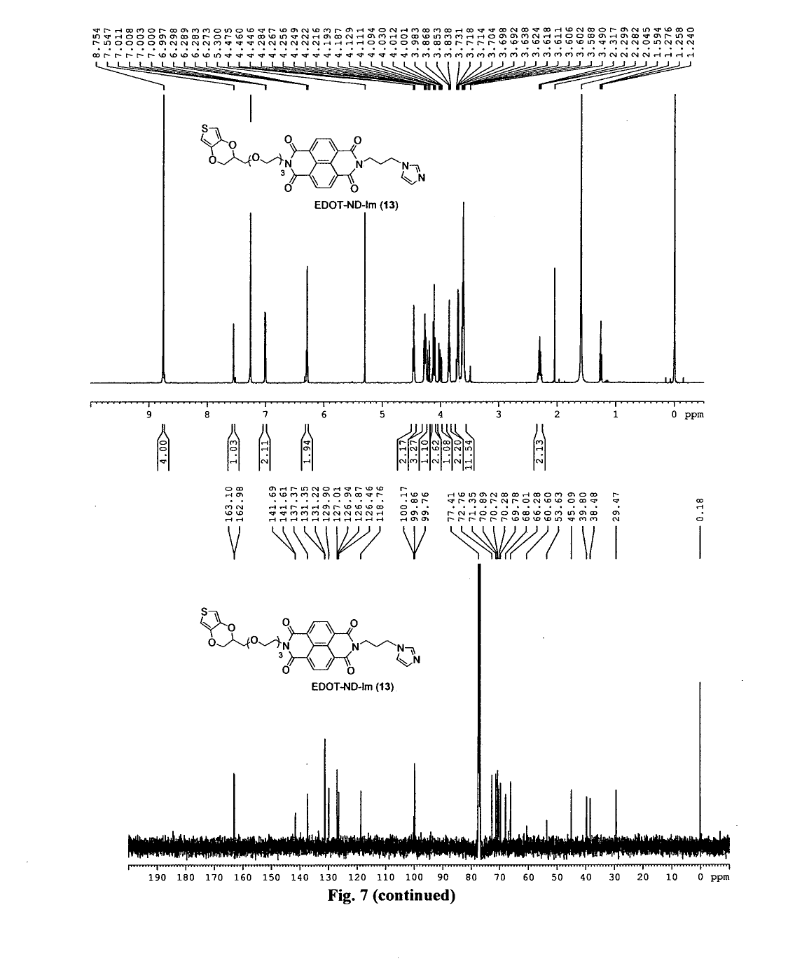

Hydroxymethyl EDOT was synthesized according to a previously described procedure ((a) A. Lima, P. Schottland, S. Sadki, C. Chevrot, Synthetic Metals 1998, 93, 33; (b) S. Akoudad, J. Roncali, Electrochemistry Communications 2000, 2, 72). All chemicals were of reagent grade and used as received. Anhydrous solvents were purchased from Sigma-Aldrich in a sure-seal bottle and introduced in the reaction flask under Ar using standard vacuum/inert gas manifold techniques. All other solvents were purchased from J. T. Baker (Phillipsburg, N.J.). All reagents were purchased from commercial sources and were used without further purification, unless indicated otherwise. Deuterated solvents were purchased from Sigma-Aldrich or Cambridge Isotope Laboratories Inc. ¹H and ¹³C NMR data was acquired at 25° C. with on a Bruker AV 400 spectrometer. NMR spectra of selected compounds are shown in FIG. 7. Flash chromatography was performed on CombiFlash Companion or Rx16 on normal phase Silicagel cartridges. MS was carried out on a Finnigan/MAT LCQ Mass Spectrometer (ThermoFinnigan, San Jose, Calif.) fitted with an ESI probe. UV-Vis spectrophotometry was performed on an Agilent 8453 diode array spectrophotometer. Peptide nucleic acid (PNA) capture probe was custom synthesized by Applied Biosystems (Foster City, Calif.), while the DNA oligonucleotides were custom-made by 1st Base Pte Ltd (Singapore). The base sequences were N-TTTGAGTCTGTTGCTTGG (linker)-Cys (PNA capture probe), 5′-CCAAGCAACAGACTCAAA (complementary DNA target) and 5′-GGTTCGTTGTCTGAGTTT (non-complementary DNA target). Electrochemical study of EDOT intercalators were performed with an Autolab PGSTAT 32 potentiostat (Metrohm) in a glovebox from Innovative Technologies (Newburyport, Mass.). The one-chamber, three-electrode cell was made up of a quasi-internal Ag wire reference electrode (CH Instruments, Inc.) submerged in 0.01 M of AgNO₃/0.1 M of (nBu)₄NPF₆ in anhydrous CH₃CN, a platinum button working electrodes, and a platinum coil counter electrode. Electrochemical DNA detection was carried out using a CH Instruments model 760C electrochemical workstation (CH Instruments, Austin, Tex.). A conventional three-electrode system, consisting of a 3.0-mm diameter gold working electrode (CH Instruments), a nonleak miniature Ag/AgCl reference electrode (Cypress Systems, Lawrence, Kans.), and a platinum wire counter electrode, was used in all electrochemical measurements. PBS buffer (15 M NaCl, 20 mM phosphate buffer; pH 7.4) was used for immobilization of PNA capture probes, while TE buffer (Tris-HCl, 1.0 mM EDTA; pH 8.0 containing 0.1 M NaCl and 0.01% triton X) was used as hybridization buffer and post-hybridization washing. Incubation of the intercalator was done in TE buffer; while a NaCl-saturated TE buffer containing 10% ethanol was used as final washing solution before analysis. Aqueous LiClO₄ solution (0.1 M) was used as electrolyte in electrochemical detection procedure.

### Example 19

### Synthesis of EDOT-ND-EDOT

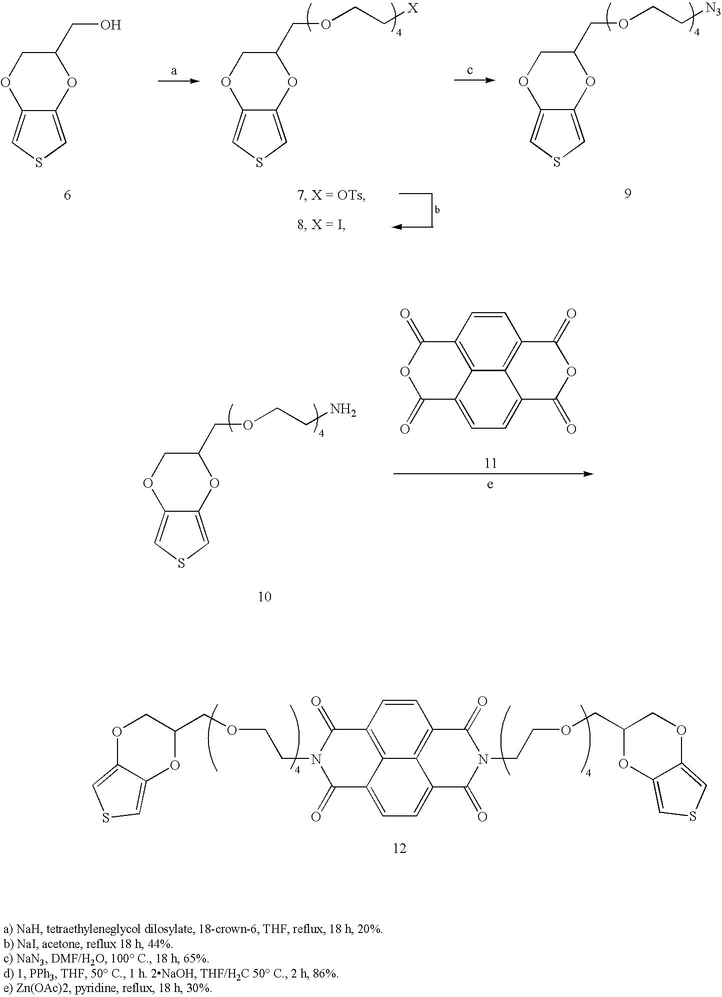

11-(2,3-dihydrothieno[3,4-b][1,4]dioxin-3-yl)methyl)-3,6,9-trioxaundecyl tosylate (EDOT-EG4-OTs, 7). A solution of EDOT-OH (6; 1.72 g, 10.0 mmol) and 18-crown-6 (132 mg, 0.500 mmol) in anhydrous THF (10 mL) was added dropwise to NaH (95%, 1.26 g, 50.0 mmol) suspended in anhydrous THF (150 mL) at 0° C. The mixture was then added to tetraethyleneglycol ditosylate (1.01 g, 20.0 mmole) and was refluxed overnight under N₂. After quenching with water, THF was removed in vacuo and extracted three times with CH₂Cl₂. The combined organic phase was dried with MgSO₄, and purified by flash chromatography (dichloromethane/ethyl acetate=9:1). EDOT-EG4-OTs (7; 1.00 g, 20%) was obtained as light yellow liquid after drying under vacuum. ¹H NMR (400 MHz, CDCl₃): δ 7.80 (d, 1H, J=1.6 Hz), 7.78 (d, 1H, J=1.2 Hz), 6.32 (d, 1H, J=3.6 Hz), 6.31 (d, 1H, J=3.6 Hz), 4.35-4.29 (m, 1H), 4.24 (dd, 1H, J=11.6, 2.0 Hz), 4.15 (t, 2H, J=4.8 Hz), 4.11 (dd, 1H, J=14.4, 7.2 Hz), 4.05 (dd, 1H, J=11.6, 7.6 Hz), 3.76 (dd, 1H, J=10.4, 4.8 Hz), 3.71-3.53 (m, 14H), 2.44 (s, 3H).

2-((11-iodo-3,6,9-trioxaundecyloxy)methyl)-2,3-dihydrothieno[3,4-b][1,4]dioxine (EDOT-EG4-I, 8). A solution of 7 (1.00 g, 1.99 mmole) and sodium iodide (1.49 g, 9.95 mmole) was refluxed in acetone (20 mL) for 18 h. The reaction mixture was then filtered, the volatiles removed in vacuo, dissolved in CH₂Cl₂, and washed with saturated Na₂S₂O_{5(aq)}. After purification by flash chromatography (dichloromethane/ethyl acetate=19:1), EDOT-EG4-I (8; 400 mg, 44%) was obtained as light yellow liquid after drying under vacuum. ¹H NMR (400 MHz, CDCl₃) δ 6.33 (d, 1H, J=3.6 Hz), 6.32 (d, 1H, J=3.6 Hz), 4.36-4.29 (m, 1H), 4.25 (dd, 1H, J=11.6, 2.8 Hz), 4.06 (dd, 1H, J=11.6, 7.2 Hz), 3.77 (dd, 1H, J=9.6, 4.8 Hz), 3.75 (t, 2H, J=5.2 Hz), 3.71-3.63 (m, 13H), 3.26 (t, 2H, J=7.2 Hz).

2-((11-azido-3,6,9-trioxaundecyloxy)methyl)-2,3-dihydrothieno[3,4-b][1,4]dioxine (EDOT-EG4-N₃, 9) A solution of 8 (400 mg, 0.873 mmole) in DMF (5 mL) and a solution of sodium azide (227 mg 3.49 mmole) in water (5 mL) were mixed together and refluxed for 18 h. DMF was removed by washing with saturated NH₄Cl_{(aq)}. The reaction mixture was dissolved in CH₂Cl₂, washed with water, and dried with MgSO₄. The crude product was purified by flash chromatography (dichloromethane/ethyl acetate=19:1) to yield a viscous colorless liquid (212 mg, 0.568 mmole, 65%). ¹H NMR (400 MHz, CDCl₃): δ 6.33 (d, 1H, J=4 Hz), 6.32 (d, 1H, J=3.6 Hz), 4.35-4.29 (m, 1H), 4.25 (dd, 1H, J=11.6, 2.4 Hz), 4.06 (dd, 1H, J=11.6, 7.2 Hz), 3.76 (dd, 2H, J=10.8, 5.2 Hz), 3.71-3.52 (m, 15H), 3.39 (t, 2H, J=5.2 Hz). ¹³C NMR (100 MHz, CDCl₃): δ 141.6, 141.5, 99.7, 99.6, 77.3, 72.6, 71.2, 70.7, 70.7, 70.6, 70.5, 70.1, 69.6, 66.1, 50.7. HR-MS (FAB): calcd. for C₁₅H₂₃N₃O₆S+H⁺ 374.1386 [M+H⁺]; found 374.1379.

2-((11-amino-3,6,9-trioxaundecyloxy)methyl)-2,3-dihydrothieno[3,4-b][1,4]dioxine EDOT-EG4-NH₂. A solution of 9 (100 mg, 0.268 mmole) in THF (3 mL) was mixed with triphenylphosphine (77.3 mg, 0.295 mmole), and was heated to 50° C. for 1 h. 3 mL of NaOH_{(aq)} (2 M) was subsequently added, and the reaction was stirred for another 2 h. THF was removed by rotary evaporator, and the aqueous reaction mixture was acidified to pH<3. The aqueous phase was washed with CH₂Cl₂. NaOH was then added, and the resulting solution (pH>10) was extracted with CH₂Cl₂. The organic layer was dried with Na₂SO₄, and the solvent was removed in vacuo to give a viscous yellow liquid (80.0 mg, 86%). ¹H NMR (400 MHz, CDCl₃): δ 6.29 (d, 1H, J=3.6 Hz), 6.28 (d, 1H, J=3.2 Hz), 4.32-4.25 (m, 1H), 4.21 (dd, 1H, J=11.6, 2 Hz), 4.02 (dd, 1H, J=11.6, 7.6 Hz), 3.72 (dd, 2H, J=10.4, 4.8 Hz), 3.67-3.57 (m, 15H), 3.47 (t, 2H, J=5.2 Hz). ¹³C NMR (100 MHz, CDCl₃): δ 141.5, 141.4, 99.7, 99.6, 72.9, 72.6, 71.1, 70.6, 70.5, 70.5, 70.2, 69.6, 66.1, 53.5, 41.5. HR-MS (FAB): calcd. for C₁₅H₂₅NO₆S+H⁺ 348.1481 [M+H⁺]; found 348.1478.

N,N′-bis[11-(2,3-dihydrothieno[3,4-b][1,4]dioxin-3-yl)methyl)-3,6,9-trioxaundecyl]-1,4,5,8-naphthalenetetracarboxydiimide (Bis-EDOT-ND, 12). Dianhydride 11 (35.1 mg, 0.131 mmol), the amine 10 (100.0 mg, 0.288 mmol), and zinc acetate (20.2 mg, 0.092 mmol) were refluxed in pyridine (10 mL) over 18 h. The reaction mixture was filtered through a short column of silica gel with CH₂Cl₂ as eluent. The organic solution was then washed with HCl (1 N) and deionized water, dried with MgSO₄, and the solvent was removed by a rotary evaporator. The diimide 12 was obtained as an orange solid (11.0 mg, 30%) after flash chromatography (dichloromethane/ethyl acetate=2:1). ¹H NMR (400 MHz, CDCl₃): δ 8.75 (s, 4H), 6.31 (d, 2H, J=3.6 Hz), 6.29 (d, 2H, J=3.6 Hz), 4.46 (t, 4H, J=6 Hz), 4.34-4.27 (m, 2H), 4.23 (dd, 2H, J=11.6, 2.4 Hz), 4.04 (dd, 2H, J=11.6, 4.8 Hz), 3.84 (t, 4H, J=5.6 Hz), 3.75 (dd, 2H, J=10.4, 4.8 Hz), 3.72-3.64 (m, 10H), 3.64-3.56 (m, 16H). ¹³C NMR (100 MHz, CDCl₃): δ 162.9, 141.5, 141.5, 131.0, 126.7, 126.6, 100.0, 99.7 99.6, 77.9, 72.6, 71.2, 70.6, 70.6, 70.5, 70.1, 69.6, 67.8, 66.1, 39.6. HR-MS (FAB): calcd. for C₄₄H₅₀N₂O₁₆S₂+H⁺ 927.2680 [M+H⁺]; found 927.2698.

### Example 20

### Synthesis of EDOT-ND-Os

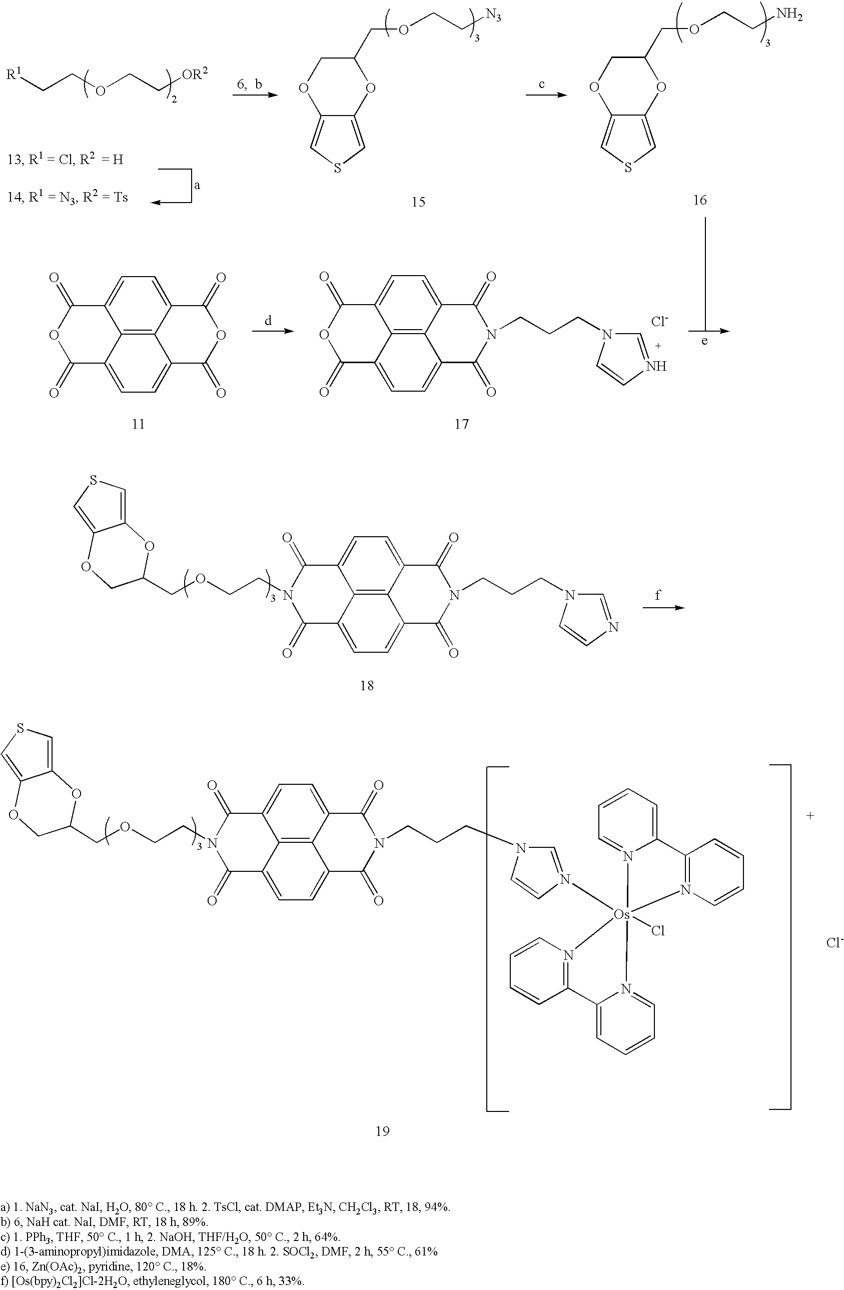

8-azido-3,6-dioxaoctyl tosylate (TsO-EG3-N₃, 14). To a solution of NaN₃ (3.25 g, 50 mmol) and NaI (0.30 g, 2 mmol), the chloride 13 (1.46 mL, 1.69 g, 10 mmol) was added and the mixture heated at 80° C. over 18 h. The solution was transferred into a separatory funnel and extracted with CH₂Cl₂ (5×). The combined organic layers were dried (MgSO₄) and the solution volume reduced to approx. 20-30 mL. Tosyl chloride (2.10 g, 11 mmol) and 4-dimethylaminopyridine (DMAP; 122 mg, 1 mmol) added, followed by dropwise addition of Et₃N (1.6 mL, 1.21 g, 12 mmol). After 18h, the solution was washed with 10% H₂SO_{3(aq)}, saturated NaHCO_{3(aq)}, dried (MgSO₄) and the volatiles removed in vacuum. The azide 14 (3.10 g, 94%) was obtained as a colourless liquid after column chromatography (CombiFlash 40 g cartridge, 0 to 60% gradient of ethyl acetate in hexane over 20 min). The ¹H and ¹³C NMR data were in agreement with those previously reported (S. J. Meunier, Q. Wu, S.-N. Wang, R. Roy, Can. J. Chem. 1997, 75, 1472).

2-((8-azido-3,6-trioxaoctyloxy)methyl)-2,3-dihydrothieno[3,4-b][1,4]dioxine (EDOT-EG3-N₃, 15). To a solution of EDOT-OH (6; 1.72 g, 10 mmol) and NaI (0.375 g, 2.5 mmol) and dry DMF (10 mL), NaH (60% in mineral oil; 600 mg, 15 mmol) were added against a weak back-flow of Ar and the mixture stirred over 20 min. A solution of 14 (3.29 g) in DMF (10 mL) was added dropwise and the mixture stirred over 18 h. The mixture was partitioned between H₂O (300 mL) and diethyl ether (100 mL) and the organic layer was further washed with H₂O (5×). After drying (MgSO4) and removal of the volatiles in vacuum, 15 (2.94 g, 89%) were obtained after column chromatography (CombiFlash 40 g cartridge, 30 to 70% gradient of ethyl acetate in hexane over 20 min). ¹H NMR (400 MHz, CDCl₃): δ 6.35 (d, 1H, J=3.6 Hz), 6.34 (d, 1H, J=3.2 Hz), 4.36-4.31 (m, 1H), 4.26 (dd, 1H, J=10.8, 1.6 Hz), 4.07 (dd, 1H, J=10.8, 7.6 Hz), 3.77 (dd, 2H, J=10.8, 5.2 Hz), 3.70-3.67 (m, 10H), 3.39 (t, 2H, J=4.8 Hz). ¹³C NMR (100 MHz, CDCl₃): δ 141.6, 141.5, 99.7, 99.6, 72.6, 71.2, 70.7, 70.6, 70.1, 69.6, 66.1, 60.4, 50.1.

2-((8-amino-3,6-dioxaoctyloxy)methyl)-2,3-dihydrothieno[3,4-b][1,4]dioxine (EDOT-EG3-NH₂, 16). A solution of 15 (660 mg, 2.0 mmol) in THF (10 mL) and triphenylphosphine (577 mg, 2.2 mmol) was heated to 50° C. for 1 h. NaOH_{(aq)} (2 M; 10 mL) was added, and the reaction mixture stirred for another 2 h. THF was removed by rotary evaporator, and the aqueous reaction mixture was acidified to pH<3. The aqueous phase was extracted with CH₂Cl₂ (3×) and the organic layers discarded. To the aqueous layer, NaOH was then added, and the solution (pH>10) was extracted with CH₂Cl₂ (3×). The organic layer was dried with Na₂SO₄, and the solvent was removed in vacuo to give a viscous yellow liquid (388 mg, 86%). ¹H NMR (400 MHz, CDCl₃): δ 6.29 (d, 1H, J=3.6 Hz), 6.28 (d, 1H, J=3.2 Hz), 4.32-4.25 (m, 1H), 4.21 (dd, 1H, J=11.6, 2 Hz), 4.02 (dd, 1H, J=11.6, 7.6 Hz), 3.72 (dd, 2H, J=10.4, 4.8 Hz), 3.67-3.57 (m, 15H), 3.47 (t, 2H, J=5.2 Hz). ¹³C NMR (100 MHz, CDCl₃): 8 141.5, 141.4, 99.7, 99.6, 72.9, 72.6, 71.1, 70.6, 70.5, 70.5, 70.2, 69.6, 66.1, 53.5, 41.5.

N-[3-(imidazol-1-yl)propyl]-N′-[8-((2,3-dihydrothieno[3,4-b][1,4]dioxin-3-yl)methoxy)-3,6-dioxaoctyl]-1,4,5,8-naphthalenetetracarboxydiimide (EDOT-ND-Im, 18). A mixture of 11 (804.6 mg, 3 mmol) and 1-(3-aminopropyl)imidazole (125.2 mg, 1 mmol) in dimethylacetate (30 mL) was heated at 125° C. for 18 hours. Upon cooling to room temperature, the of CHCl₃ (100 mL) was added. The precipitate was filtered, and the volatiles removed in vacuum. Water (150 mL) was added and precipitate formed was washed with ethanol and ether. The crude product was heated with SOCl₂ (142.8 mg, 1.2 mmol) in DMF at 60° C. for 2 hours. The precipitate formed was collected and washed with ether to afford 17 (251.2 mg, 0.61 mmol, 61%), which was used for the next step without further purification. A mixture of 16 (50 mg, 0.17 mmol), 17 (70 mg, 0.17 mmol) and zinc acetate (25.4 mg, 0.12 mmol) was heated in pyridine at 120° C. for 15 h. After removal of pyridine by vacuum distillation, the reaction mixture was partitioned between CH₂Cl₂ and Na. The combined organic layer was dried with MgSO₄ and subjected to further purification by flash chromatography (dichloromethane/ethyl acetate=1/9). The product was obtained as yellow gel (18.5 mg, 0.03 mmol, 17.6% yield). ¹H NMR (400 MHz, CDCl₃): δ 8.74 (t, 4H, J=8 Hz), 7.56 (m, 1H), 7.02 (m, 2H), 6.32 (d, 1H, J=3.6 Hz), 6.31 (d, 1 H, J=3.6 Hz), 4.48 (t, 2H, J=6 Hz), 4.32-4.25 (m, 1H), 4.23 (dd, 1H, J=11.6, 2 Hz), 4.12 (t, 2H, J=7.6 Hz), 4.05 (dd, 1H, J=11.6, 7.2 Hz), 3.87 (dd, 1H, J=10.4, 6 Hz), 3.71 (t, 2H, J=5.6 Hz), 3.66-3.60 (m, 8 H), 2.32 (q, 2H, J=5.6 Hz), ¹³C NMR (400 MHz, CDCl₃): δ 163.1. 163.0, 141.7, 141.6, 137.4, 131.4, 131.2, 129.9, 127.0, 126.9, 126.9, 126.5, 118.8, 100.2, 99.8, 72.8, 71.4, 70.9, 70.7, 70.3, 69.8, 68.0, 66.3, 45.1, 39.8, 38.5, 29.5.

N[3-(imidazol-1-yl)propyl]-N′-[8-((2,3-dihydrothieno[3,4-b][1,4]dioxin-3-yl)methoxy)-3,6-dioxaoctyl]-1,4,5,8-naphthalenetetracarboxydiimide complex with Os(bpy)₂Cl₂ (EDOT-NTCDI-Os (19). [Os(bpy)₂Cl₂]Cl.2H₂O^{[3]} (18.9 mg, 0.029 mmol) was added to a solution of 18 (18.5 mg, 0.030 mmol) in ethylene glycol, and the mixture was stirred at 180° C. over 6 hours. The progress of the reaction was monitored by cyclic voltammetry. Upon completion, ethylene glycol was removed and the solid was extracted with CHCl₃ (3×). The combined CHCl₃ layers were washed repeatedly with water. The product was obtained as dark purple paste (14.2 mg, 33% yield).

### Example 21

### Electrochemical Experiments and DNA Detection.

Immobilization of PNA capture probe (CP) on gold electrode. The gold button electrode was first mechanically polished with 0.5 μm alumina slurry, followed by ultrasonication in IPA (isopropanol) and water. Electrochemical cleaning was subsequently done by repeated potential scanning at −0.3 to 1.5 V (vs. Ag/AgCl) in 0.1 M H₂SO₄ solution. Upon washing in water and drying with a stream of nitrogen, the electrode was ready to use. A monolayer of CP was adsorbed by immersing the gold electrode in a 1 μM solution of CP in PBS (phosphate buffered saline) for 12 h. After adsorption, the electrode was copiously rinsed with PBS and soaked in and blown dry with a stream of nitrogen. To minimize non-specific uptake of target DNA and intercalator, and improve the quality and stability of the CP monolayer, the CP-coated gold electrode was immersed in an ethanolic solution of 1 mM 11-mercaptoundecanol (MUD) for 3 h. Unreacted MUD was rinsed with ethanol and then with water. Upon blow drying with nitrogen, the electrode was ready for the next step.

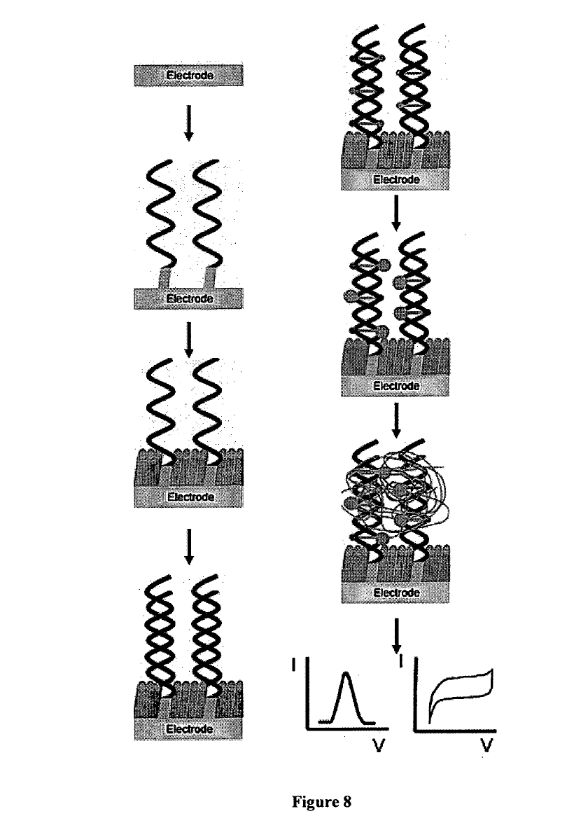

Hybridization and detection. The hybridization of target DNA was done in a moisture-saturated chamber maintained at 37° C. A 2.5 μl aliquot of hybridization solution containing the target DNA was uniformly spread onto the CP-coated electrode and left to hybridize for 4 hours. To remove non-specifically bound DNA from the electrode surface, a series of high- and low-stringency washes was carried out. The electrode was first washed in a stirred hybridization buffer (blank, with 0.01% triton-X) at 37° C. for 5 minutes, followed by immersion in blank TE buffer at room temperature for 1 minute, and finally a brief wash in water. A 3.0 μl aliquot of 100 μM EDOT-ND-Os in the TE buffer was then added to the electrode surface, allowing it to incubate at room temperature for 15 min. After being thoroughly rinsed with the final washing solution, the electrode was ready for the electrochemical analysis (FIG. 8). First, the redox reaction of Os⁺²/Os⁺³ was observed through square wave voltammetry in 0.1 M solution of LiClO_{4(aq)} (results are shown in FIG. 6A). Next, the electrode was subjected to five cycles of potential scan at −0.2 to 1.0 V (vs. Ag/AgCl) to form oligoEDOTs. These serve as ‘seeds’ for subsequent polymerization. In the final step, the electrode was immersed in a 0.1 M solution of LiClO_{4(aq)} containing 5.0 mM of EDOT-OH. A constant potential was applied at 0.9 V (vs. Ag/AgCl) for 120 seconds to allow polymerization of the EDOT-OH monomers. After a brief washing in water, the electrode was analyzed in a blank electrolyte solution to quantify the copolymer film that has been formed (result shown in FIG. 6C).

1. A compound of structure (I) 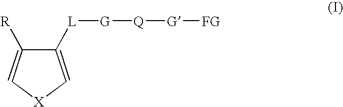
wherein:
X is S, O or NR^{N}, where R^{N} is H or alkyl;
L is a linker group;
Q is a species capable of binding with dsDNA;
G and G′ are, independently, absent or have between 1 and 30 main chain atoms;
FG is a functional moiety comprising at least one O or N atom or a transition metal complex; and
R is selected from the group consisting of H, alkyl, alkoxy or OCR^{a}R^{b} coupled to an atom in L so as to form a six-membered ring, wherein R^{a} and R^{b} are independently H or optionally substituted alkyl.

2. The compound of claim 1 wherein X is S.

3. The compound of claim 1 or claim 2 wherein Q is capable of intercalating the dsDNA.

4. The compound of claim 3 wherein Q is a naphthalene diimide group.

5. The compound of any one of claims 1 to 4 wherein L has structure OCH₂.

6. The compound of any one of claims 1 to 4 wherein the compound has structure (II):

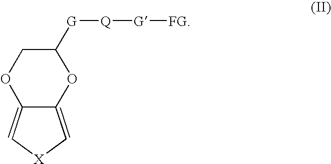

7. The compound of any one of claims 1 to 6 wherein G and G′ are independently selected from the group consisting of CH₂, (CH₂)₃, CH₂O(CH₂)₆, CH₂OCH₂(CH₂OCH₂)₂CH₂, CH₂OCH₂(CH₂OCH₂)₄CH₂ and CH₂OCH₂(CH₂OCH₂)₃CH₂.

8. The compound of any one of claims 1 to 7 wherein FG is OH, NH₂, imidazolyl, pyridinyl or a metal complex.

9. The compound of any one of claims 1 to 7 wherein FG has structure (III) 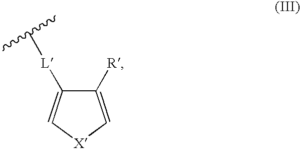
where L′ is a linker group, X′ is S, O or NR^{N}, where R^{N} is H or alkyl, and R′ is selected from the group consisting of H, alkyl, alkoxy or OCR^{c}R^{d} coupled to an atom in L′ so as to form a six-membered ring, wherein R^{c} and R^{d} are independently H or optionally substituted alkyl.

10. The compound of claim 9 wherein X′ is S.

11. The compound of claim 9 or claim 10 wherein L′ has structure OCH₂.

12. The compound of any one of claims 9 to 11 wherein the compound has structure (IV):

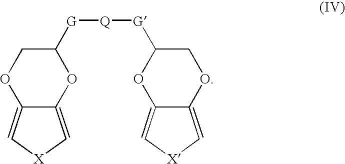

13. The compound of claim 12 wherein G and G′ are the same and X and X′ are the same.

14. An electrically conducting polymer comprising monomer units derived from a compound according to any one of claims 1 to 13.

15. The polymer of claim 14, said polymer being a copolymer and additionally comprising monomer units derived from a second monomer, said second monomer being an optionally substituted thiophene, an optionally substituted pyrrole or an optionally substituted furan, or a mixture of any two or more of these, wherein said optionally substituted thiophene, optionally substituted pyrrole or optionally substituted furan is unsubstituted in the 2 and 5 positions.

16. The copolymer of claim 15 wherein said second monomer is a 3,4-ethylenedioxythiophene.

17. A process for making a compound of structure (I) as defined in claim 1, said process comprising reacting a compound of structure (V) 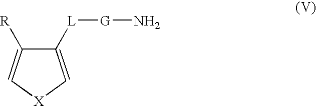
and a compound of structure FG-G′-NH₂ with a compound of structure A-Q-A, wherein A is a functional group capable of coupling with an amine group and Q is a group capable of binding with dsDNA.

18. The process of claim 17 wherein the compound of structure A-Q-A is naphthalene dianhydride.

19. The process of claim 17 or claim 18 wherein the compound of structure FG-G′-NH₂ has structure (V).

20. A compound of structure (I) as defined in claim 1 when made by the process of any one of claims 17 to 19.

21. A process for making a polymer according to claim 14, said process comprising electropolymerising a monomer of structure (I) as described in claim 1.

22. The process of claim 21 wherein the monomer of structure (I) is mixed with a second monomer, said second monomer being an optionally substituted thiophene, an optionally substituted pyrrole or an optionally substituted furan, wherein said optionally substituted thiophene, optionally substituted pyrrole or optionally substituted furan is unsubstituted in the 2 and 3 positions.

23. A polymer according to claim 14 when made by the process of claim 21 or claim 22.

24. A method for determining the presence or absence of a dsDNA in a sample comprising:
exposing the sample to a compound according to any one of claim 1 to 13 or 20, or to a polymer according to any one of claim 14 to 16 or 23,
comparing a signal from the compound or polymer before said exposing to a corresponding signal from the compound or polymer after said exposing, and
determining the presence or absence of a dsDNA in the sample from said comparing.

25. The method of claim 24 wherein the signal is selected from the group consisting of absorbance of UV/visible light, absorbance maximum of UV/visible light, electrical impedance, electrical resistance, electrical conductivity and onset potential for electrical conductivity

26. A sensor for detecting the presence or absence of dsDNA in a sample, said sensor comprising an electrically conducting polymer according to any one of claim 14 to 16 or 23.

27. A method for determining the presence or absence of a specific polynucleotide sequence in a sample comprising:
providing an electrode having bonded to the surface thereof a polynucleotide sequence complementary to said specific nucleotide sequence;
exposing the electrode to the sample and to a compound according to any one of claim 1 to 13 or 20;
supplying a cyclic voltage to the electrode so as to electropolymerise said compound to form a conducting polymer;
measuring a cyclic voltammogram of the conducting polymer on the electrode;
comparing said voltammogram with the voltammogram of a control electrode, and
determining the presence or absence of the specific polynucleotide sequence in the sample from said comparing.

28. A method for determining the presence or absence of a specific polynucleotide sequence in a sample comprising:
providing an electrode having bonded to the surface thereof a polynucleotide sequence complementary to said specific nucleotide sequence;
exposing the electrode to the sample;
supplying a cyclic voltage to the electrode in the presence of a compound according to any one of claim 1 to 13 or 20 so as to electropolymerise said compound to form a conducting polymer;
measuring a cyclic voltammogram of the conducting polymer on the electrode;
comparing said voltammogram with the voltammogram of a control electrode; and
determining the presence or absence of the specific polynucleotide sequence in the sample from said comparing.

29. The method of claim 27 or 28 wherein said method is a method for determining a concentration of said specific polynucleotide sequence, wherein the step of comparing comprises comparing the magnitude of a current in said voltammogram with the magnitude of a current in a voltammogram measured using a known concentration of said specific nucleotide sequence and wherein the step of determining comprises determining the concentration of the specific polynucleotide sequence in the sample from the comparing.
